(19) United States
(12) Patent Application Publication
(10) Pub. No.: US 2026/0283545 A1
(43) Pub. Date: Sep. 24, 2026

(54) SYSTEMS, DEVICES, AND METHODS FOR EVALUATING AND PREDICTING SLEEP-RELATED DISORDERS

(71) Applicant: RESMED DIGITAL HEALTH INC., San Diego, CA (US)

(72) Inventors: Jonathon Marlon LOBBINS, Irvine, CA (US); Mojgan PAYOMBAR, Menlo Park, CA (US); Nicolas Gabriel ARCINO, Berkeley, CA (US); Sonja RADOVANCEVIC, South San Francisco, CA (US); Rosemary KU, San Francisco, CA (US)

(73) Assignee: RESMED DIGITAL HEALTH INC., San Diego, CA (US)

(21) Appl. No.: 19/168,757

(22) PCT Filed: Mar. 21, 2024
(86) PCT No.: PCT/US2024/020971

§ 371 Date: Sep. 24, 2025

Related U.S. Application Data
(60) Provisional application No. 63/454,599, filed on Mar. 24, 2023.
(60) Provisional application No. 63/543,888, filed on Oct. 12, 2023.

(51) Int. Cl. A61B 5/00; A61B 5/0205; A61B 5/024; A61B 5/145; A61B 5/16

(52) U.S. Cl. CPC A61B 5/4815; A61B 5/0205; A61B 5/02405; A61B 5/14542; A61B 5/165; A61B 5/4809; A61B 5/4812; A61B 5/4818; A61B 5/7275; A61B 5/743; A61B 5/744

(57) ABSTRACT

Aspects of this disclosure relate to evaluating and predicting sleep related disorders. In some variations, a user health graphic is generated on a graphical user interface. For example, a plurality of shapes may be generated based on a respective sleep, physical health, and mental health rating. A location of the plurality of shapes may be arranged based on the sleep, physical health, and mental health rating. In some variations user data comprising a sleep rating, a physical health rating, and a mental health rating, as well as sleep measurement data, are received and risk of one or more sleep disorders are predicted based on the user data and the sleep measurement data.

### CROSS-REFERENCE TO RELATED APPLICATIONS

This application is a 371 national phase application of PCT Application No. PCT/US2024/020971 filed on Mar. 21, 2024, which claims the benefit of U.S. Provisional Application No. 63/454,599, filed Mar. 24, 2023, and U.S. Provisional Application No. 63/543,888, filed Oct. 12, 2023, the content of each of which is hereby incorporated by reference in its entirety.

### TECHNICAL FIELD

Devices, systems, and methods herein relate to remotely evaluating and predicting a medical condition, including but not limited to sleep-related disorders.

### BACKGROUND

Millions of people suffer from chronic medical conditions, including sleep-related disorders such as sleep apnea, orthopnea, and insomnia. Chronic medical conditions cost the U.S. healthcare system on average about seven thousand dollars per year per person per condition in direct costs where sleep disorders alone account for about seven thousand dollars per year per person in direct costs to the U.S. healthcare system. About 47% of all insured Americans have multiple chronic medical conditions. As such, it may be desirable to provide new and useful ways to monitor and proactively address chronic medical conditions.

### SUMMARY

Described here are systems, devices, and methods for remotely evaluating and predicting a medical condition such as a sleep-related disorder. These systems and methods may, for example, receive user input data and sleep measurement data, and analyze the received data for trends and/or a risk of a sleep disorder. In some variations, a user health graphic may be generated on a graphical user interface based on user data to graphically represent user health. This may, for example, allow insight into the health and well-being of a user on a continuous or semi-continuous, real-time basis.

In some variations, a method of graphically representing user health may comprise outputting a notification using a computing device for user input of a sleep rating, a physical health rating, and a mental health rating, and generating a user health graphic on a graphical user interface of the computing device. The generating may comprise generating a first shape comprising a first color based on the sleep rating, a second shape comprising a second color based on the physical health rating, and a third shape comprising a third color based on the mental health rating, and arranging a location of the first shape, the second shape, and the third shape relative to a first axis of the user health graphic based on the sleep rating, the physical health rating, and the mental health rating.

In some variations, the first axis may be a longitudinal axis of the user health graphic. In some variations, the first shape may comprise a first center, the second shape may comprise a second center, and the third shape may comprise a third center. The first center, the second center, and the third center may be positioned on the first axis.

In some variations, the first shape may comprise a first center, the second shape may comprise a second center, and the third shape may comprise a third center and two of the first center, the second center, and the third center may be positioned on the first axis, and the third of the first center, the second center, and the third center may be offset from the first axis.

In some variations, the first shape may comprise a first center, the second shape may comprise a second center, and the third shape may comprise a third center and one of the first center, the second center, and the third center may be positioned on the first axis, and the other two of the first center, the second center, and the third center may be offset from and on opposing sides of the first axis. In some variations, the first axis may be an angled axis between a longitudinal axis of the user health graphic and a lateral axis of the user health graphic. In some variations, a center-to-center distance between the first shape and the second shape may be based on a differential between two of the sleep rating, the physical health rating, and the mental health rating.

In some variations, centers of two or more of the first shape, the second shape, and the third shape may be spaced apart from each other along the first axis. In some variations, centers of two or more of the first shape, the second shape, and the third shape may be offset from each other relative to the first axis. In some variations, the first shape may comprise a first center, the second shape may comprise a second center, and the third shape may comprise a third center and the first center, the second center, and the third center may be laterally offset from each other. In some variations, the first shape, the second shape, and the third shape may partially overlap along the first axis. In some variations, the first shape may comprise a first center, the second shape may comprise a second center, and the third shape may comprise a third center and the first center, the second center, and the third center may be spaced apart by an equal center-to-center distance along the first axis.

In some variations, generating the user health graphic may comprise generating a plurality of lines, where each line of the plurality of lines overlaps two of the first shape, the second shape, and the third shape. In some variations, the plurality of lines may comprise a first line between a center of the first shape and a center of the second shape, and a second line between the center of the second shape and a center of the third shape.

In some variations, the sleep rating, the physical health rating, and the mental health rating may each correspond to a predetermined scale. In some variations, the predetermined scale may be a Likert scale.

In some variations, each of the first shape, the second shape, and the third shape may comprise a two-dimensional geometry. In some variations, each of the first color, the second color, and the third color may comprise a color gradient. In some variations, each of the first color, the second color, and the third color may comprise an opacity gradient. In some variations, a background of the graphical user interface may comprise a color gradient.

In some variations, the method may further comprise periodically updating the user health graphic. In some variations, the method may further comprise generating an animation comprising the updated user health graphic and one or more previous user health graphics. In some variations, the notification may be output at a predetermined interval.

In some variations, the method may further comprise modifying computing device settings based on the user health graphic. In some variations, the method may further comprise referring a sleep service to a user based on the user health graphic.

Also described here are methods of predicting a sleep disorder. In some variations, a method of predicting a sleep disorder may include receiving user data comprising a sleep rating, a physical health rating, and a mental health rating from a computing device and sleep measurement data from a measurement device, predicting a risk of one or more sleep disorders based on the user data and the sleep measurement data, and referring a sleep service to a user based on the predicted risk of the one or more sleep disorders.

In some variations, the method may further comprise outputting a notification for input of the user data using the computing device. In some variations, the sleep measurement data may comprise one or more sleep parameters. In some variations, the one or more sleep parameters may comprise one or more of a sleep start time, a sleep end time, a sleep duration, a time to fall asleep, a number of times woken, a wake period duration, a snore status, a snore duration, an exercise duration, an exercise end time, a number of alcoholic drinks consumed, a time of last alcoholic drink consumed, a number of caffeine drinks consumed, a time of last caffeine drink consumed, a daytime sleepiness status, a daytime sleepiness severity, a daytime sleepiness status, a daytime sleepiness severity, an average sleeping oxygen saturation, an average sleeping heart rate variability, a minimum sleeping heart rate, a maximum sleeping heart rate, an average sleeping heart rate, a set of medications consumed, and user demographic data. In some variations, the one or more sleep parameters may further comprise a user wake time note and a user bedtime note. In some variations, the user demographic data may comprise one or more of medical history and test results.

In some variations, the sleep rating, the physical health rating, and the mental health rating may each correspond to a predetermined scale. In some variations, the predetermined scale may be a Likert scale. In some variations, the sleep rating may comprise a sleep quality status, the physical health rating may comprise a body status, and the mental health rating may comprise an emotional status. In some variations, the sleep quality status may comprise a wake reason. In some variations, the body status may comprise a body discomfort type a body discomfort area, and a body discomfort intensity. In some variations, the emotional feeling status may comprise a wake time emotion and a bedtime emotion.

In some variations, the method may further comprise generating sleep data based on the user data and the sleep measurement data. In some variations, generating sleep data may comprise reconciling the user data and the sleep measurement data. In some variations, the method may further comprise generating one or more sleep trends by analyzing the sleep data. In some variations, the risk of one or more sleep disorders may be based on the one or more sleep trends.

In some variations, the method may further comprise generating a graphical user interface comprising one or more of the sleep trend and the risk of sleep disorder. In some variations, the method may further comprise modifying computing device settings based on one or more of the sleep trend and the risk of sleep disorder. In some variations, the sleep service referral may be based on one or more of the sleep trend and the risk of sleep disorder. In some variations, the user data and the sleep measurement data may be received at a predetermined interval.

In some variations, the predetermined interval may be at least once daily. In some variations, the predetermined interval may be at wake time and bedtime. In some variations, the sleep disorder may comprise one or more of obstructive sleep apnea, central sleep apnea, hypoapnea, orthopnea, nocturnal atrial fibrillation, nocturnal hypertension, nocturnal discomfort, evening exacerbated chronic obstructive pulmonary disease, heart failure, asthma, sleep quality, and waking discomfort.

Also described here are methods of graphically representing user health that may include receiving user data comprising a sleep rating, a plurality of physical health parameters, and a mental health rating from a computing device and sleep measurement data from a measurement device. A sleep quality rating may be generated based on the sleep rating and the sleep measurement data, a physical health quality rating based on the plurality of physical health parameters, and a mental health quality rating based on the mental health rating. A user health graphic may be generated on a graphical user interface of the computing device, and may include generating a first shape based on the sleep quality rating, a second shape based on the physical health quality rating, and a third shape based on the mental health quality rating. A location of the first shape, the second shape, and the third shape may be arranged relative to a first axis of the user health graphic based on the sleep quality rating, the physical health quality rating, and the mental health quality rating.

In some variations, the method may further comprise periodically generating the sleep quality rating, the physical health quality, and the mental health quality rating. In some variations, the sleep measurement data may comprise one or more of sleep duration, sleep efficiency, sleep latency, a number of awakenings, time spent awake, and sleep quality. In some variations, the sleep quality rating may be based on a weighted sum of the sleep rating and the sleep measurement data. In some variations, the weighting may be based on time.

In some variations, the plurality of physical health parameters may comprise one or more of a physical health rating, a number of physical discomforts, a number of maximum value physical discomforts, and an average severity of physical discomfort. In some variations, the physical health quality rating may be based on a weighted sum of the plurality of physical health parameters. In some variations, the weighting may be based on time. In some variations, the plurality of physical health parameters may each correspond to a predetermined scale. In some variations, the predetermined scale may be a Likert scale.

In some variations, generating the user health graphic may comprise an alphanumeric representation of the sleep quality rating, the physical health quality rating, and the mental health quality rating. In some variations, the user data and the sleep measurement data may be received at a predetermined interval. In some variations, the predetermined interval may be at least once daily.

In some variations, the first axis may be a longitudinal axis of the user health graphic. In some variations, the first shape may comprise a first center, the second shape may comprise a second center, and the third shape may comprise a third center, where the first center, the second center, and the third center may be positioned on the first axis. In some variations, the first shape may comprise a first center, the second shape may comprise a second center, and the third shape may comprise a third center. Two of the first center, the second center, and the third center may be positioned on the first axis, and the third of the first center, the second center, and the third center may be offset from the first axis.

In some variations, the first shape may comprise a first center, the second shape may comprise a second center, and the third shape may comprise a third center. One of the first center, the second center, and the third center may be positioned on the first axis, and the other two of the first center, the second center, and the third center may be offset from and on opposing sides of the first axis.

In some variations, the first axis may be an angled axis between a longitudinal axis of the user health graphic and a lateral axis of the user health graphic. In some variations, a center-to-center distance between the first shape and the second shape may be based on a differential between two of the sleep rating, the physical health rating, and the mental health rating.

In some variations, centers of two or more of the first shape, the second shape, and the third shape may be spaced apart from each other along the first axis. In some variations, centers of two or more of the first shape, the second shape, and the third shape may be offset from each other relative to the first axis.

In some variations, the first shape may comprise a first center, the second shape may comprise a second center, and the third shape may comprise a third center. The first center, the second center, and the third center may be laterally offset from each other. In some variations, the first shape, the second shape, and the third shape may partially overlap along the first axis. In some variations, the first shape may comprise a first center, the second shape may comprise a second center, and the third shape may comprise a third center. The first center, the second center, and the third center may be spaced apart by an equal center-to-center distance along the first axis.

In some variations, generating the user health graphic may comprise generating a plurality of lines. Each line of the plurality of lines may overlap two of the first shape, the second shape, and the third shape. In some variations, the plurality of lines may comprise a first line between a center of the first shape and a center of the second shape, and a second line between the center of the second shape and a center of the third shape. In some variations, each of the first shape, the second shape, and the third shape may comprise a two-dimensional geometry. In some variations, each of the first color, the second color, and the third color may comprise a color gradient. In some variations, each of the first color, the second color, and the third color may comprise an opacity gradient.

In some variations, a background of the graphical user interface may comprise a color gradient. In some variations, the method may further include periodically updating the user health graphic. In some variations, an animation may be generated comprising the updated user health graphic and one or more previous user health graphics. In some variations, a notification may be output for input of the user data using the computing device. The notification may be output at a predetermined interval.

In some variations, the method may further include modifying computing device settings based on the user health graphic. In some variations, the method may further include referring a sleep service to a user based on the user health graphic.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a block diagram of a variation of a sleep evaluation system.

FIG. 2 is a block diagram of a variation of a computing device.

FIG. 3A is an illustrative flow chart of variations of a sleep disorder prediction process.

FIG. 3B is an illustrative flow chart of variations of a first assessment process. FIG. 3C is an illustrative flow chart of variations of a second assessment process.

FIG. 4 is an illustrative flow chart of variations of a process for graphically representing user health status.

FIGS. 5A-5D are a set of illustrative variations of a wake time graphical user interface.

FIGS. 6A-6D are a set of illustrative variations of an objective sleep data graphical user interface.

FIGS. 7A-7H are a set of illustrative variations of a subjective sleep data graphical user interface.

FIG. 8 is an illustrative variation of a wake time note graphical user interface.

FIGS. 9A and 9B are a set of illustrative variations of a bedtime graphical user interface.

FIGS. 10A-10G are a set of illustrative variations of a sleep insight graphical user interface.

FIGS. 11A and 11B are a set of illustrative variations of a sleep disorder prediction graphical user interface.

FIG. 12 is an illustrative variation of an actionable suggestion graphical user interface.

FIG. 13 is an illustrative variation of a referral graphical user interface.

FIGS. 14A and 14B are a set of illustrative variations of a health status report.

FIGS. 15A and 15B are a set of illustrative variations of a user health graphic.

FIG. 16 is a schematic diagram of a variation of a key for a user health graphic.

FIGS. 17A-17AA are another set of illustrative variations of a user health graphic.

FIG. 18 is an illustrative variation of a user health graphical user interface.

FIG. 19 is another illustrative variation of a user health insight graphical user interface.

### DETAILED DESCRIPTION

Described here are systems, devices and methods for providing an actionable suggestion to a user in predicting a risk of a chronic condition such as a sleep disorder. These systems, devices and methods may, for example, obtain sleep data from one or more of the user and a measurement device(s) for analysis and/or display, and generation of trends, insights, and predictions that may be presented to the user and/or a set of predetermined contacts (e.g., health care professional, family, friends, caregiver) along with one or more suggestions that the user may take action on in view of the trends, insights, and predictions. This may, for example, give the user a holistic understanding of their health and provide a tangible step towards improvement. For some users, review of their sleep data and trends may be complex and burdensome. However, the systems and methods described herein may provide intuitive insight (e.g., plain language summary) into the sleep data and trends. For example, a user health status represented by a set of parameters (e.g., sleep, body, mind) may be a graphically visualized on a graphical user interface (GUI) in order to facilitate a holistic and intuitive understanding of a user's health. In some variations, an actionable suggestion may be generated based on the data and/or trends. The suggestion may include one or more steps that the user, the computing device, and/or a referred service (e.g., health care professional) may perform for user health.

Sleep provides a valuable opportunity to monitor and evaluate sleep-related and non-sleep related health conditions. A sleeping state, waking state, and/or lying state may provide a high signal-to-noise ratio for symptoms of many non-sleep health conditions (e.g., heart failure, COPD). For example, decreased external stimuli at bedtime and/or during sleep (e.g., reduced light, activity) may facilitate user awareness of symptoms of health conditions that may be present throughout the day. Accordingly, sleep tracking (e.g., self-reported sleep-adjacent symptoms, multi-modal sensor measurements) may be useful for tracking overall health. Furthermore, some sleep-related disorders have a high overlap with many non sleep-related disorders. For example, obstructive sleep apnea and insomnia may be comorbid with a range of chronic physical health conditions and mental behavioral health conditions. Sleep disturbances in general may be indicative of chronic physical and/or mental health conditions (e.g., early morning waking is a sign of depression).

Generally, the systems and methods described herein may receive data from a measurement device (e.g., sleep tracker, cardiac monitor, activity tracker) configured to measure one or more user health characteristics during and/or associated with sleep. The data generated from each of these devices may be automatically uploaded to a user's computing device (e.g., smartphone, laptop, PC) and/or a database (e.g., cloud based storage) at predetermined intervals. The data may be reconciled (e.g., integrated) with user data (e.g., subjective user input sleep data) for analysis of trends and/or generation of insights. For example, trends may be presented to the user on any device (e.g., sleep measurement device, smartphone, laptop) with selectable levels of complexity (e.g., detailed, concise, summary, plain language, long-term, medium-term, short-term). Presenting the trends to the user in an accessible and/or customizable manner may increase the user's understanding of how behavior (e.g., sleep, physical health, mental health) correlates with health. A health care professional may also be granted access to the user sleep data and trends data.

Furthermore, the systems and methods may generate an actionable suggestion in response to one or more of the sleep data, trends, and predictions. The suggestion may include an action that the user may perform themselves (e.g., reduce caffeine and/or alcohol consumption, set an evening sleep routine) and/or an action to be performed by a computing device (e.g., present sleep disorder information, encourage healthy behavior). The actionable suggestion may be output on a computing device as a prompt that the user may select to confirm execution of the actionable suggestion In some variations, the suggested action may be executed automatically without user input. In some variations, the actionable suggestion may be based on observations derived from user data (e.g., user input subjective sleep data), sleep measurement data, and trends. In some variations, the actionable suggestion may include a referral to a sleep service, which may be useful when the user has a predicted high risk of a sleep disorder.

Generally, the systems described herein comprise one or more of a computing device and a sleep measurement device. The sleep measurement device may be configured to generate measurement data (e.g., sleep data, cardiac data) that may be transmitted for processing and analysis. Data analysis may include trend analysis to find relationships between user data (e.g., subjective user input data) and the sleep data measured by the measurement device, as well as relationships using derived values such as the sleep quality ratings, physical health quality ratings, and mental health quality ratings. Trend analysis of different data sets (e.g., user data, sleep measurement data, sleep quality ratings, physical health quality ratings, and mental health quality ratings) associated with one or more of sleep, physical health, and mental health may provide holistic insight to a user's overall health. The results of the analysis may be used to generate one or more prompts to output to the user on a graphical user interface. For example, data analysis showing that a user is exhibiting a trend (e.g., high risk) for a sleep disorder (e.g., sleep apnea) may be used to output a user prompt referring them to a health care professional, and/or to add an actionable suggestion (e.g., reduce caffeine consumption) to promote a desired behavior (e.g., higher quality sleep). The user may receive a prompt from the system to modify the settings of the computing device (e.g., add reminders, measure additional parameters) in response to one or more of the data analysis, trend analysis, and/or predictions. As another example, data analysis showing that a user is on a positive trend may be used to generate a prompt providing positive reinforcement to the user.

As used herein, a specific output and any data or signal corresponding thereto will be referred to as a “prompt.” A specific user input and any data or signal corresponding thereto will be referred to as a “command.” In some variations, the prompt may suggest a command for the user. For example, a prompt may output a command (e.g., actionable suggestion, recommendation, observation) that a user may affirmatively input to a device. For example, a prompt may be displayed on a user's device (e.g., a touch screen of a computing device) and may suggest that the user discuss their predicted risk of a sleep disorder with a health care professional along with display of a prompt suggesting to schedule an appointment with their health care professional (e.g., “Do you wish to consult a specialist?”). The user may confirm execution of the prompt to schedule an appointment by entering a selection into the device (e.g., selecting a corresponding icon on the touch screen of the computing device) thereby providing a command.

### I. Systems

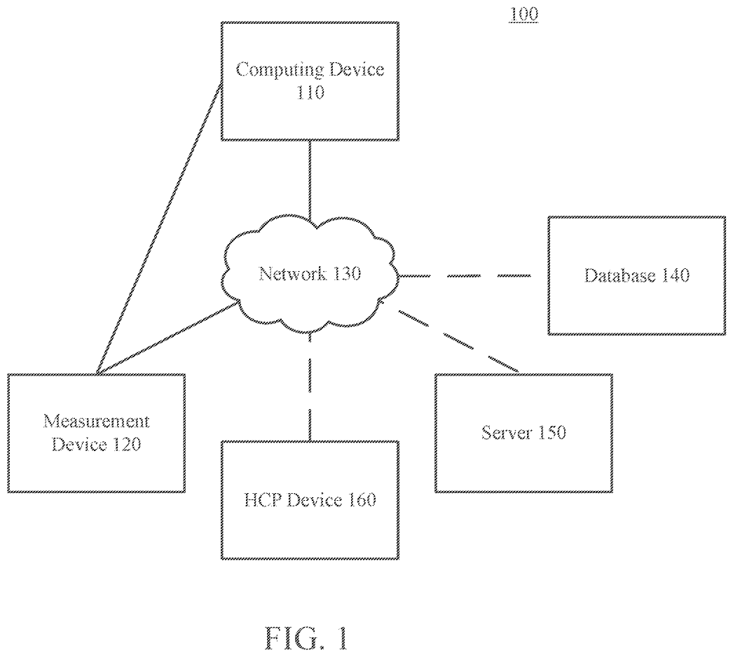

A sleep evaluation system may include one or more of the components necessary to measure and analyze user data using the devices as described herein. In addition, the system may be configured to generate and output a prediction and/or a prompt to encourage healthy behavior based on the data and/or analysis thereof. FIG. 1 is a block diagram of a variation of a sleep evaluation system (100). The system (100) may comprise a computing device (110) (e.g., a user device) and a measurement device (120) (e.g., a sleep measurement device) configured to measure user one or more sleep parameters such as sleep start time, sleep end time, sleep duration, fall asleep minutes, wake count, sleep quality, and as further described in more detail herein. The computing device (110) and the measurement device (120) may be communicably coupled through one or more wired or wireless communication channels. For example, the computing device (110) and the measurement device (120) may be coupled to one or more of a network (130), a database (140), a server (150), and a health care professional (HCP) device (160) through one or more wired or wireless communication channels. Processing and analysis may be performed at any one of the devices of the system (100) or distributed throughout a plurality of devices.

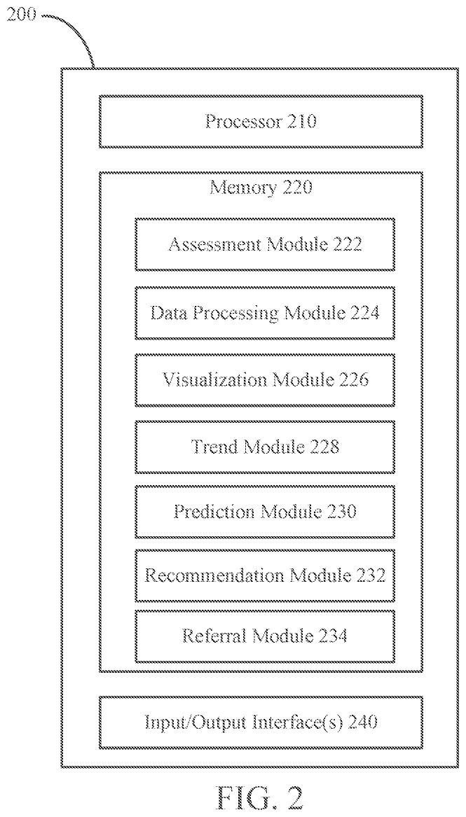

FIG. 2 is a block diagram of a variation of a computing device (200). For example, the computing device (200) may correspond to the computing device (110) of the sleep evaluation system (100) described with respect to FIG. 1. While a single computing device is schematically depicted, it can be appreciated that the systems described herein may include a plurality (e.g., two, three, four, five, or more) computing devices. In some variations, the computing device (200) (or any other device of the system (100)) may be configured to evaluate and predict a sleep disorder, and may provide one or more of an actionable suggestion and a referral to a sleep service. The computing device (200) may include an integration of a full-stack technology including one or more of raw data collection from a set of devices (e.g., computing device, measurement device, server, database), data storage, feature extraction, and analysis to identify trends and predict a risk of a sleep disorder.

The computing device (200) may include a processor (210), memory (220), and at least one input/output interface (240). The memory (220) may be configured to store instructions associated with one or more of an assessment module (222) configured for receiving user data, a data processing module (224) configured to process raw data, a visualization module (226) configured to generate a visual graphic, a trend module (228) configured to generate a sleep trend, a prediction module (230) configured to predict a risk of a sleep disorder, a recommendation module (232) configured to generate an actionable suggestion, and a referral module (234) configured to refer a sleep service.

In some variations, the assessment module (222) may be configured to receive user input data in response to a set of sleep and/or health-related questions from one or more of the computing devices and the sleep measurement devices described herein. The questions may be based on a set of clinically recognized sleep questionnaires as described herein. In some variations, the assessment module (222) may be configured to output a notification to a user at predetermined intervals for user input of user data including, for example, a sleep rating, a physical health rating, and a mental health rating. In some variations, the sleep rating, the physical health rating, and the mental health rating may each correspond to a predetermined scale. For example, the predetermined scale may be a Likert scale (e.g., 3 point scale, 5 point scale, 10 point scale, n point scale). In some variations, the assessment module (222) may be configured to receive sleep measurement data from one or more of a measurement device and data source (e.g., database 140, server 150, HCP device 160).

In some variations, the sleep rating may comprise a sleep quality status, the physical health rating may comprise a body status, and the mental health rating may comprise an emotional status. In some variations, the sleep quality status may comprise a wake reason. In some variations, the body status may comprise a body discomfort type a body discomfort area, and a body discomfort intensity. In some variations, the emotional feeling status may comprise a wake time emotion and a bedtime emotion.

In some variations, the user data and the sleep measurement data may be received at a predetermined interval. For example, the predetermined interval may be at least once daily, at least twice daily, at least three time daily, at least four times daily, at least five times daily, between once daily and twice daily, between once daily and three times daily, between once daily and four times daily, between once daily and five times daily, between twice daily and three times daily, between twice daily and four times daily, and between twice daily and five times daily. In some variations, the predetermined interval may correspond with particular times during the day. For example, in some variations, the predetermined interval may be at a wake time and bedtime as set by a user.

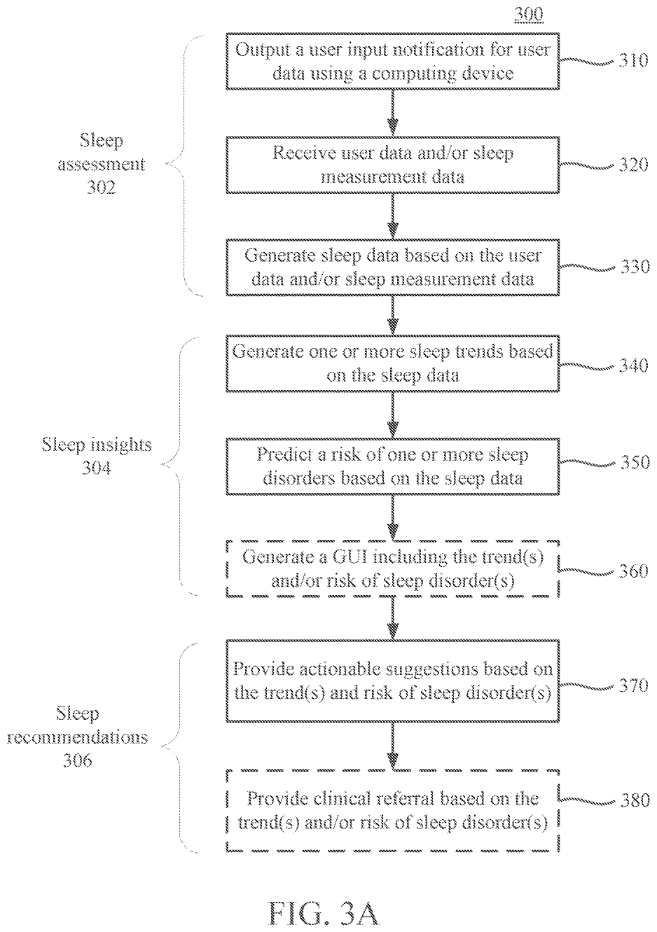

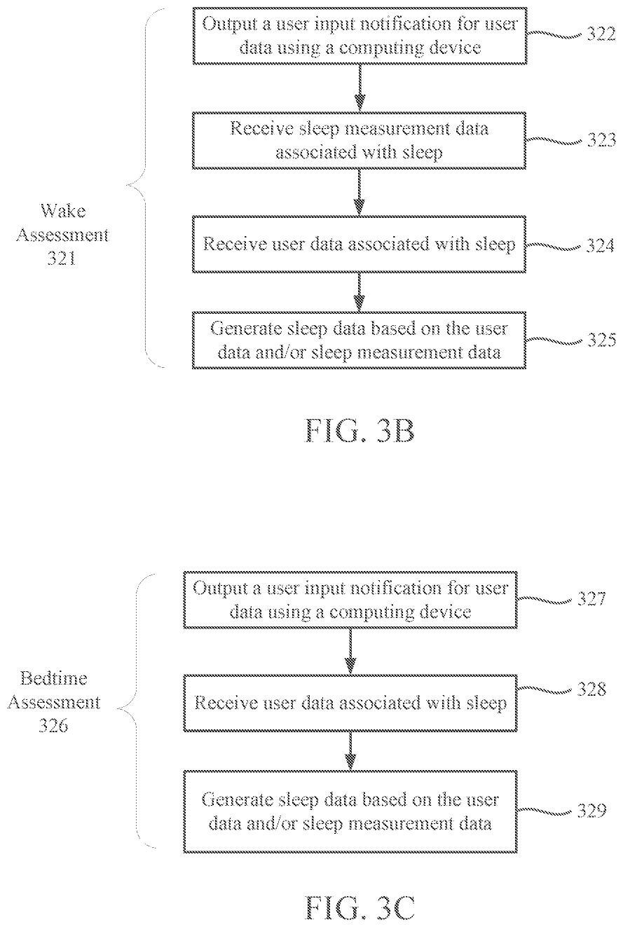

In some variations, the user data and the sleep measurement data may be integrated into user data comprising one or more sleep parameters, For example, a sleep parameter may comprise one or more of a sleep start time, a sleep end time, a sleep duration, a sleep stage, a time to fall asleep, a number of times woken, a wake period duration, a snore status, a snore duration, an exercise duration, an exercise end time, a number of alcoholic drinks consumed, a time of last alcoholic drink consumed, a number of caffeine drinks consumed, a time of last caffeine drink consumed, a daytime sleepiness status, a daytime sleepiness severity, a daytime sleepiness status, a daytime sleepiness severity, an average sleeping oxygen saturation, an average sleeping heart rate variability, a minimum sleeping heart rate, a maximum sleeping heart rate, an average sleeping heart rate, a set of medications consumed, and user demographic data. In some variations, the user demographic data may comprise one or more of medical history and test results. In some variations, a sleep parameter may further comprise a user's wake time notes and a user's bedtime notes. The processes associated with assessing user health are described in more detail herein (e.g., FIGS. 3A-3C and 5A-9B).

In some variations, a data processing module (226) may be configured to process raw data (e.g., user data, sleep measurement data) from one or more of the computing devices and the sleep measurement devices described herein to generate sleep data. In some variations, data processing may include one or more of filtering, outlier removal, missing value handling, and data integration (e.g., reconciliation). For example, a user may reconcile (e.g., edit, correct, update) one or more measured sleep parameters such as wake time, wake count, or any of the other sleep parameters described herein to ensure data integrity and generate sleep data. In some variations, the data processing module (226) may be configured to output processed sleep data that may be input to one or more of the trend module (228), the prediction module (230), the recommendation module (232), the referral module (234), and the visualization module (224).

In some variations, a visualization module (226) may be configured to generate a graphical representation of user health (e.g., user health graphic) based on one or more of user data, sleep measurement data, and/or sleep data as described herein. In some variations, generating a user health graphic may include generating a user health graphic on a graphical user interface of a computing device, and generating a first shape comprising a first color based on the sleep rating, a second shape comprising a second color based on the physical health rating, and a third shape comprising a third color based on the mental health rating. A location of the first shape, the second shape, and the third shape may be arranged relative to a first axis of the user health graphic based on the sleep rating, the physical health rating, and the mental health rating.

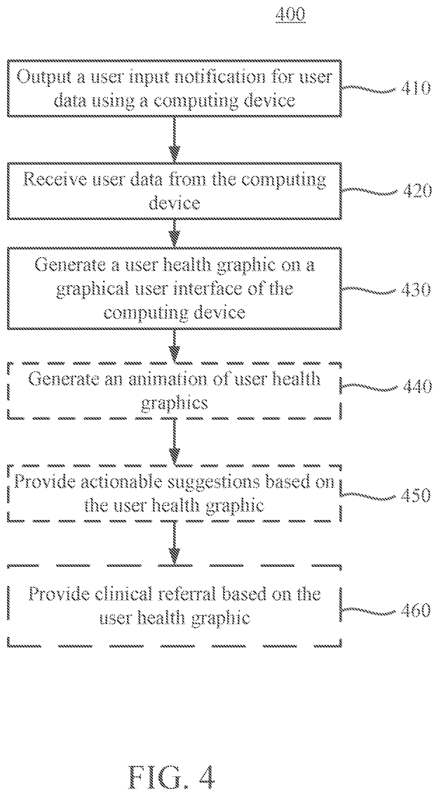

In some variations, a center-to-center distance between the first shape and the second shape may be based on a differential between two of the sleep rating, the physical health rating, and the mental health rating. In some variations, the first shape, the second shape, and the third shape may partially overlap. For example, the first shape, the second shape, and the third shape may overlap along the first axis (or any axis). In some variations, generating the user health graphic may optionally comprise generating a plurality of lines overlapping at least two of the first shape, the second shape, and the third shape. In some variations, each of the first shape, the second shape, and the third shape may comprise a two-dimensional geometry. In some variations, each of the first color, the second color, and the third color may comprise a color gradient. In some variations, each of the first color, the second color, and the third color may comprise an opacity gradient. In some variations, the plurality of lines may comprise a first line between a center of the first shape and a center of the second shape, and a second line between the center of the second shape and a center of the third shape. The processes associated with graphically representing user health are described in more detail herein (e.g., FIGS. 4, 15A-17AA).

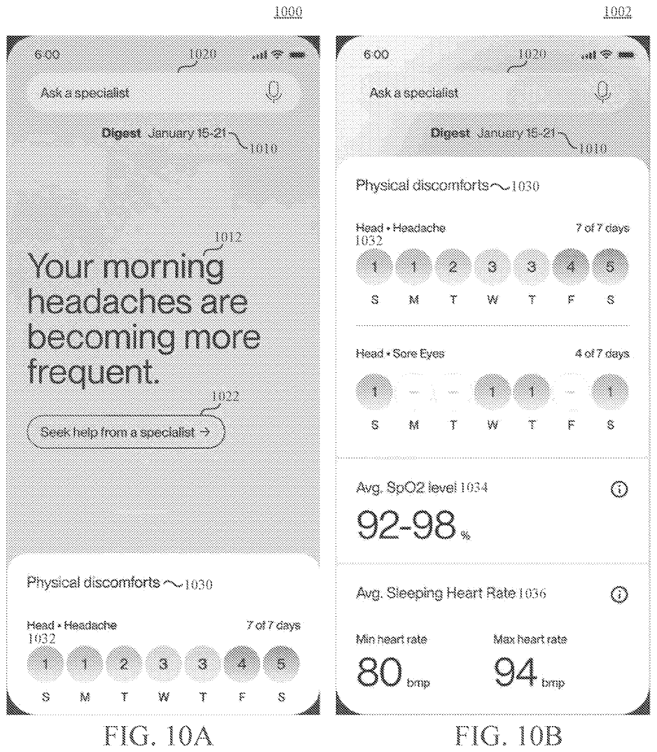

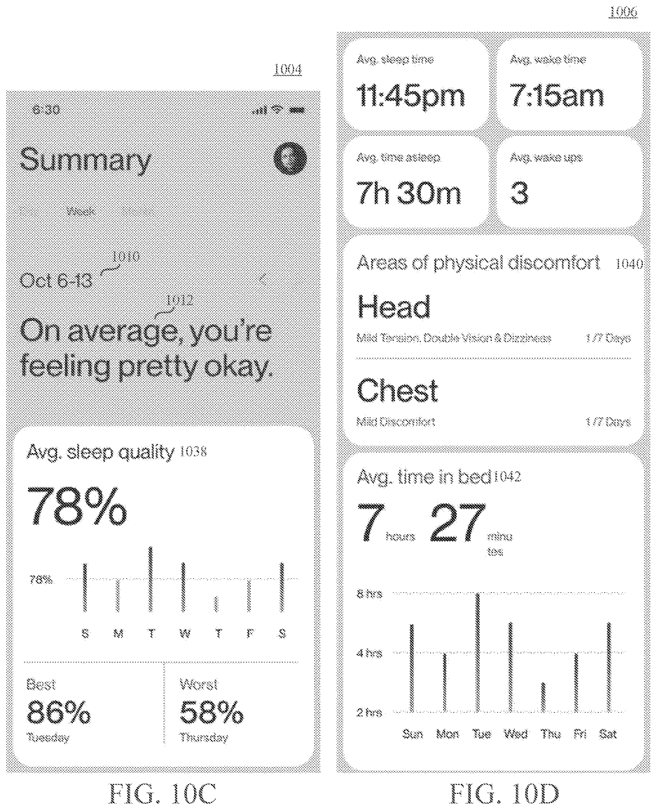

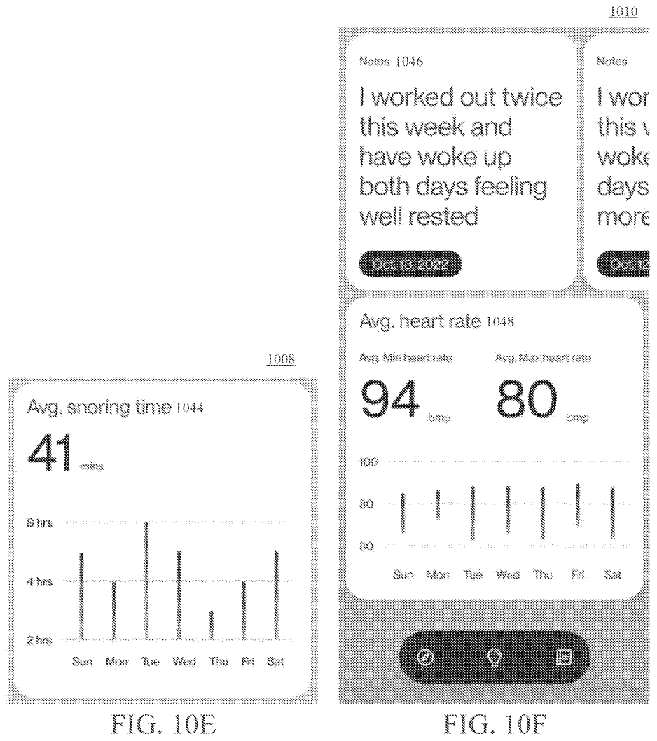

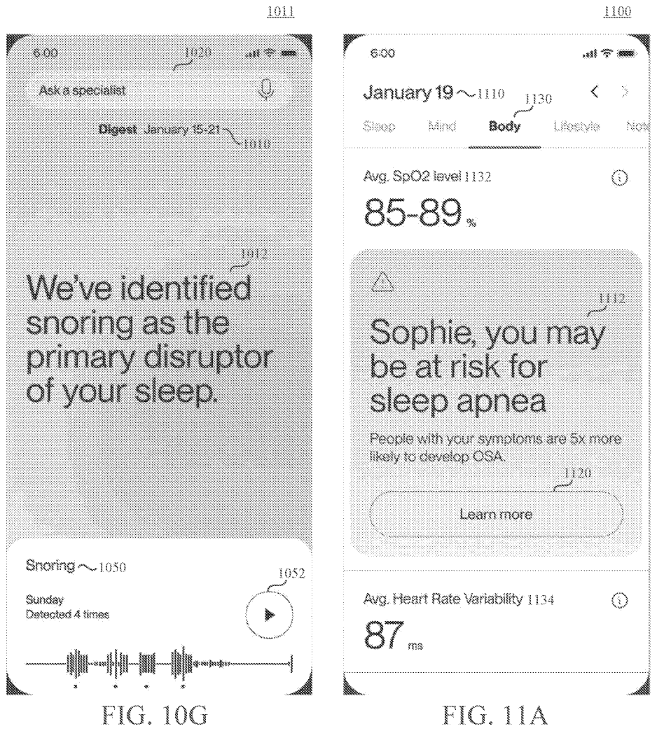

In some variations, the trend module (228) may be configured to generate one or more sleep trends by analyzing the sleep data generated by the data processing module (224). Furthermore, the trend module (228) may be configured to generate a graphical user interface comprising one or more of the sleep trend and the risk of one or more sleep disorders. The processes associated with generating a trend are described in more detail herein (e.g., FIGS. 10A-10G).

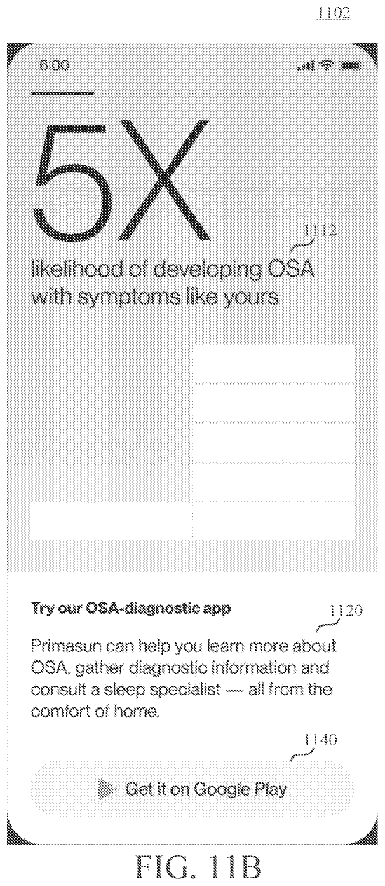

In some variations, the prediction module (230) may be configured to predict a risk of one or more sleep disorders based on the user data and the sleep measurement data. In some variations, the risk of one or more sleep disorders may be based on the one or more sleep trends. In some variations, a graphical user interface may be generated based on one or more of the sleep trend and the risk of one or more sleep disorders. In some variations, the one or more sleep disorders may comprise one or more of obstructive sleep apnea, central sleep apnea, hypoapnea, orthopnea, nocturnal atrial fibrillation, nocturnal hypertension, nocturnal discomfort, evening exacerbated chronic obstructive pulmonary disease, heart failure, asthma, sleep quality, and waking discomfort. The processes associated with generating a prediction are described in more detail herein (e.g., FIGS. 11A-11B). In some variations, the prediction module (230) may include one or more machine learning models as described in more detail herein.

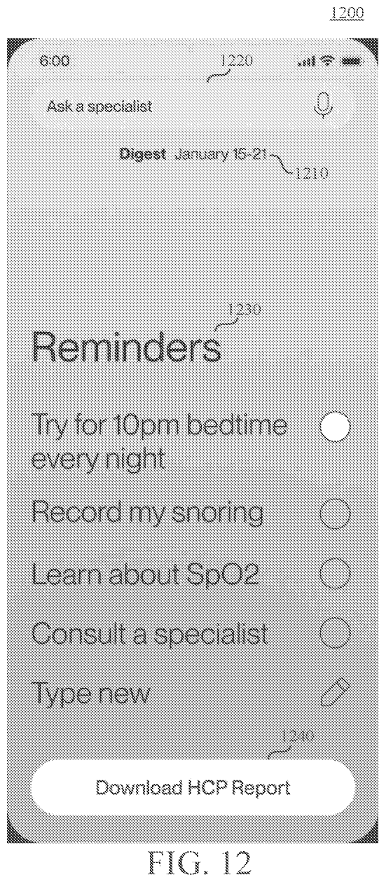

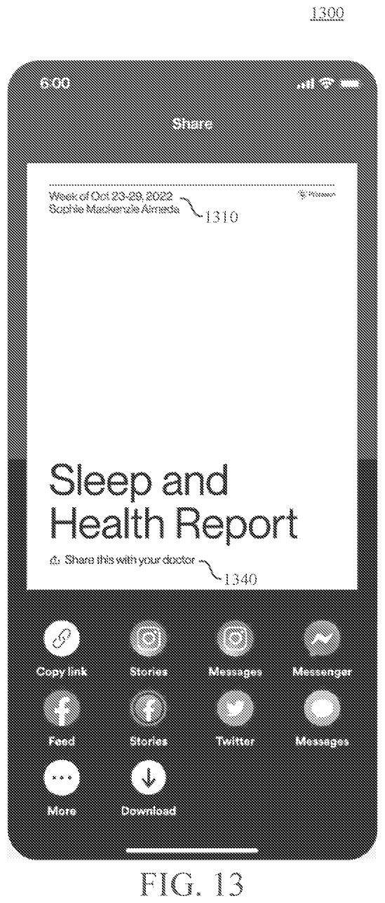

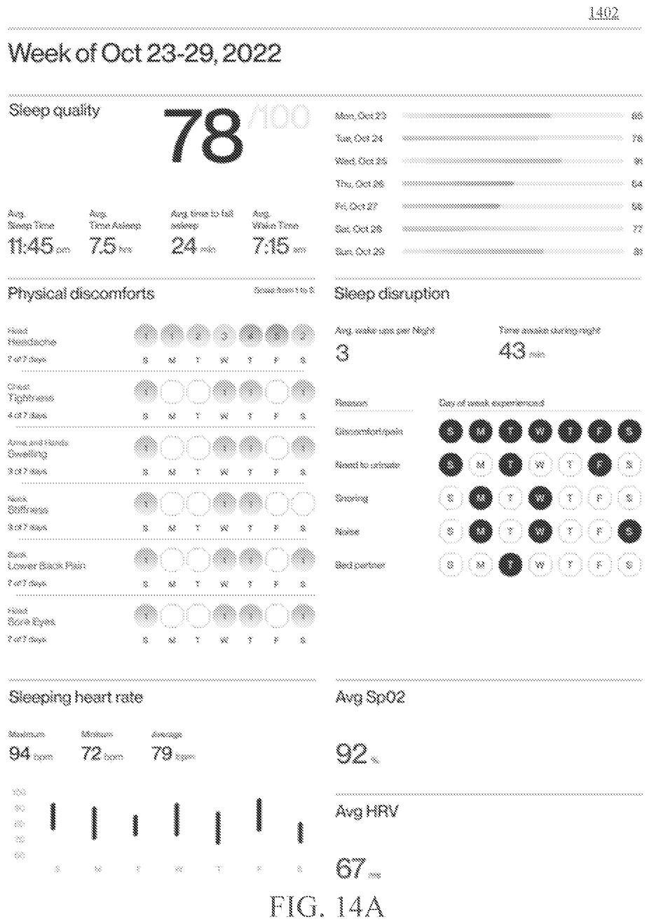

In some variations, the recommendation module (232) may be configured to generate one or more actionable suggestions. In some variations, an actionable suggestion may comprise modifying computing device settings based on one or more of the user data, sleep measurement data, processed data, and the user health graphic. The processes associated with generating a recommendation are described in more detail herein (e.g., FIG. 12).

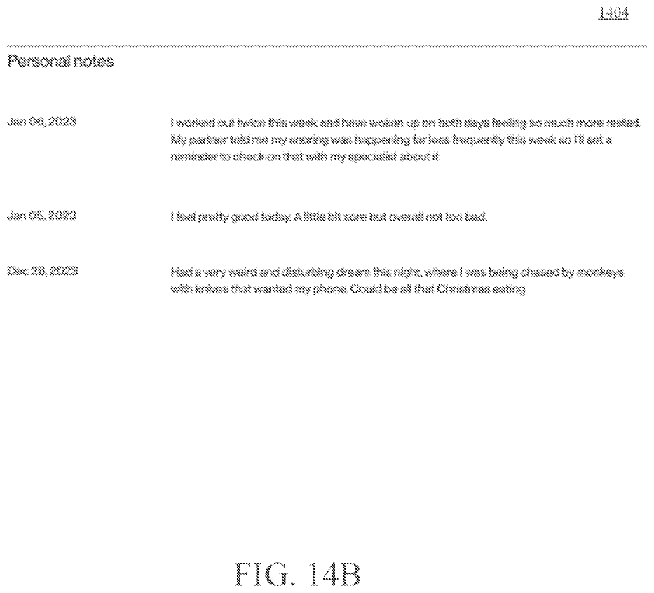

In some variations, the referral module (234) may be configured to refer a sleep service to a user using the computing device based on one or more of a sleep trend and a risk of the one or more sleep disorders. The processes associated with generating a referral are described in more detail herein (e.g., FIGS. 13-14B).

### Measurement Device

A measurement device as used herein may refer to any device configured to measure, receive, and/or analyze one or more characteristics of a user. A measurement device may, for example, measure a sleep parameter, user activity, and/or nutrition. Non-limiting examples of measurement devices include a sleep tracker, a wearable activity device (e.g., pedometer or other activity tracker), a hydration tracker, a blood pressure monitor, a heart rate monitor, an ultrasonic (e.g., sonar) sensor, a cholesterol monitor, a scale, geolocation devices (e.g., GPS, GLONASS), a smartphone, a refrigerator, a PC, an implantable diagnostic device, an ingestible diagnostic device, and other diagnostic devices. The measurement device may include one or more sensors configured to measure one or more user parameters. Sensor measurable parameters generally may include, but are not limited to, sleep, hydration, cholesterol, oxygen saturation, carbon dioxide saturation, pH, respiratory rate, respiratory sounds, vocal sounds (e.g., cough), ultrasonic audio, accelerometer with step counting and/or positional data, impedance, resistance, temperature, air quality, weight, blood pressure, heart rate, heart rate variability, and the like. In some variations, ultrasonic audio (e.g., sonar data) may be generated by a sonar system configured to emit an ultrasonic audio signal and receive a reflected ultrasonic audio signal. Furthermore, a sleep parameter may comprise one or more of a sleep start time, a sleep end time, a sleep duration, a time to fall asleep, a number of times woken, a wake period duration, a snore status, audio, a snore duration, an exercise duration, an exercise start time, an exercise end time, a number of alcoholic drinks consumed, a time of last alcoholic drink consumed, a number of caffeine drinks consumed, a time of last caffeine drink consumed, a daytime sleepiness status, a daytime sleepiness severity, an average sleeping oxygen saturation, an average sleeping heart rate variability, a minimum sleeping heart rate, a maximum sleeping heart rate, an average sleeping heart rate, a set of medications consumed, and user demographic data. For example, a snore status may indicate if a user snored during a sleep period.

Measurement data generated by the measurement device may include, but is not limited to, duration of sleep, quality of sleep, mood/feeling, stress, nutrition data (e.g., meal marking, consumed carbohydrate count, consumed calorie count, etc.), activity or exercise (e.g., calories burned, steps performed, degree or intensity of activity (e.g., based on heart rate levels), duration of activity, etc.), user weight, oral or other medications, hydration, and the like. The data generated by the measurement device may be transmitted to any of the devices of the system (100), and may include one or more of the features, elements, and/or functionality of the computing devices, as described herein. For example, the measurement device may comprise a controller comprising a processor and memory to perform data analysis on the measurement data generated by the measurement device and a communication interface configured to transmit the measurement data to another device. The measurement device may couple to a device using any known wired or wireless connection method and communication protocol.

Furthermore, while in some variations the measurement device may be a wearable device as described above, it should be understood that in other variations, the measurement device may be configured as a non-wearable device. One example of a non-wearable measurement device for measuring one or more sleep parameters is an implantable device or an external monitor such as a bedside monitor or home virtual assistant device (e.g., similar to Amazon Echo® or Google Home™ devices), a set top box service (e.g., similar to Apple TV®), or other smart appliances such as a clock, radio, and the like. In some variations, systems described herein may comprise a plurality of measurement devices, one or more of which may be a wearable device and one or more of which may be configured as a non-wearable device.

### Computing Devices

Generally, the computing devices described herein may comprise a controller comprising a processor (e.g., CPU) and a memory (which can include one or more computer-readable storage mediums). The processor may incorporate data received from memory and user input to control one or more components of the system (e.g., measurement device (120), database (140), server (150), HCP device (160)). The memory may further store instructions to cause the processor to execute modules, processes and/or functions associated with the methods described herein. As used herein, a computing device may refer to any of the computing devices (110), databases (140), servers (150), and HCP devices (160) as depicted in FIG. 1. In some variations, the memory and the processor may be implemented on a single chip. In other variations, they can be implemented on separate chips.

The computing device (110, 200) may be configured to receive and process user input to the computing device and measurement data from one or more measurement devices. The computing device may be configured to receive, compile, store, and access data. In some variations, the computing device may be configured to access and/or receive data from different sources. The computing device may be configured to receive data directly input by a user and/or it may be configured to receive data from separate devices (e.g., a measurement device, a smartphone, a tablet, a computer) and/or from a storage medium (e.g., a flash drive, a memory card). The computing device may receive the data through a network connection, as discussed in more detail herein, or through a physical connection with the device or storage medium (e.g. through Universal Serial Bus (USB) or any other type of port). The computing device may include any of a variety of devices, such as a cellular telephone (e.g., smartphone), tablet computer, laptop computer, desktop computer, portable media player, wearable digital device (e.g., digital glasses, wristband, wristwatch, brooch, armbands, virtual reality/augmented reality headset, jewelry (e.g., bracelet, necklace, ring)), television, set top box (e.g., cable box, video player, video streaming device), gaming system, or the like.

The computing device may be configured to receive various types of data. For example, the computing device may be configured to receive personal data (e.g., gender, weight, birthday, age, height, diagnosis date, anniversary date using the device, etc.), user data (e.g., sleep rating, a physical health rating, and a mental health rating), nutrition data (e.g., what a user had to eat each day, number of alcoholic beverages, amount of carbohydrates consumed, etc.), activity data (e.g., if a user exercised, when the user exercised, duration of exercise, what type of exercise the user completed (e.g. biking, swimming, running, etc.), exertion level of the exercise (e.g., low, medium, high), heart rate during exercise, etc.), measurement data from a measurement device, or any other information. In some variations, the computing device may be configured to create, receive, and/or store user profiles. A user profile may contain any of the user specific information previously described.

While the above-mentioned information may be received by the computing device, in some variations, the computing device may be configured to calculate any of the above data from information it has received using software stored on the device itself, or externally. In some variations, the computing device may be configured to compare the subjective data, measured data, nutrition data, activity data, or any other relevant data, to historical data (e.g., user's historical trends), data preloaded onto the computing device that has been compiled from external sources (e.g. other devices), or data received from a set of separate devices (e.g., historical data or data compiled from external sources).

The processor may be any suitable processing device configured to run and/or execute a set of instructions or code and may include one or more data processors, image processors, graphics processing units, physics processing units, digital signal processors, and/or central processing units. The processor may be, for example, a general purpose processor, Field Programmable Gate Array (FPGA), an Application Specific Integrated Circuit (ASIC), and/or the like. The processor may be configured to run and/or execute application processes and/or other modules, processes and/or functions associated with the system and/or a network associated therewith. The underlying device technologies may be provided in a variety of component types (e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and/or the like.

In some variations, the memory may include a database (not shown) and may be, for example, a random access memory (RAM), a memory buffer, a hard drive, an erasable programmable read-only memory (EPROM), an electrically erasable read-only memory (EEPROM), a read-only memory (ROM), Flash memory, and the like. The memory may store instructions to cause the processor to execute modules, processes, and/or functions associated with the communication device, such as measurement data processing, measurement device control, communication, and/or device settings. Some variations described herein relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also may be referred to as code or algorithm) may be those designed and constructed for the specific purpose or purposes.

Examples of non-transitory computer-readable media include, but are not limited to, magnetic storage media such as hard disks, floppy disks, and magnetic tape; optical storage media such as Compact Disc/Digital Video Discs (CD/DVDs); Compact Disc-Read Only Memories (CD-ROMs), and holographic devices; magneto-optical storage media such as optical disks; solid state storage devices such as a solid state drive (SSD) and a solid state hybrid drive (SSHD); carrier wave signal processing modules; and hardware devices that are specially configured to store and execute program code, such as Application-Specific Integrated Circuits (ASICs), Programmable Logic Devices (PLDs), Read-Only Memory (ROM), and Random-Access Memory (RAM) devices. Other variations described herein relate to a computer program product, which may include, for example, the instructions and/or computer code disclosed herein.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including C, C++, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

In some variations, the computing device (110) may further comprise a communication interface configured to permit a user to control one or more of the devices of the system. The communication interface may comprise a network interface configured to connect the computing device to another system (e.g., Internet, remote server, database) by wired or wireless connection. In some variations, the computing device (110) may be in communication with other devices via one or more wired and/or wireless networks. In some variations, the network interface may comprise a radiofrequency receiver, transmitter, and/or optical (e.g., infrared) receiver and transmitter configured to communicate with one or more devices and/or networks. The network interface may communicate by wires and/or wirelessly with one or more of the measurement device (120), network (130), database (140), server (150), and HCP device (160).

The network interface may comprise RF circuitry may receive and send RF signals. The RF circuitry may convert electrical signals to/from electromagnetic signals and communicate with communications networks and other communications devices via the electromagnetic signals. The RF circuitry may comprise well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth.

Wireless communication through any of the computing and measurement devices may use any of plurality of communication standards, protocols and technologies, including but not limited to, Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (WiFi) (e.g., IEEE 802.11a, IEEE 802.11b, IEEE 802.11g, IEEE 802.11n, and the like), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol. In some variations, the devices herein may directly communicate with each other without transmitting data through a network (e.g., through NFC, Bluetooth, WiFi, RFID, and the like).

The communication interface may further comprise a user interface configured to permit a user (e.g., user, predetermined contact such as a partner, family member, health care professional, coach, etc.) to control the computing device. The communication interface may permit a user to interact with and/or control a computing device directly and/or remotely. For example, a user interface of the computing device may include an input device for a user to input commands and an output device for a user to receive output (e.g., trends, insights, prompts on a display device).

An output device of the user interface may output data analysis and actionable prompts corresponding to the user and may comprise one or more of a display device and audio device. For example, a video conference between the user and a health care professional may be facilitated using the display device of the computing device. A display device may permit a user to view trend analysis, predictions, insights, and/or other data processed by the controller. Data analysis generated by the server (150) may be displayed by the output device (e.g., display) of the computing device (110). Measurement data from one or more measurement devices (120) may be received through the network interface and output visually and/or audibly through one or more output devices of the computing device (110). In some variations, an output device may comprise a display device including at least one of a light emitting diode (LED), liquid crystal display (LCD), electroluminescent display (ELD), plasma display panel (PDP), thin film transistor (TFT), organic light emitting diodes (OLED), electronic paper/e-ink display, laser display, and/or holographic display.

An audio device may audibly output user data, measurement data, predictions, system data, alarms and/or notifications. For example, the audio device may output an audible alarm when an assessment is not performed by the user within a predetermined time period (e.g., morning assessment performed within fifteen minutes of a regular wake-up time). In some variations, an audio device may comprise at least one of a speaker, piezoelectric audio device, magnetostrictive speaker, and/or digital speaker. In some variations, a user may communicate with other users using the audio device and a communication channel. For example, a user may form an audio communication channel (e.g., VoIP call) with a remote health care professional.

In some variations, the user interface may comprise an input device (e.g., touch screen) and output device (e.g., display device) and be configured to receive input data from one or more of the measurement devices (120), network (130), database (140), server (150), and HCP device (160). For example, user control of an input device (e.g., keyboard, buttons, touch screen) may be received by the user interface and may then be processed by processor and memory for the user interface to output a control signal to one or more measurement devices (120). Some variations of an input device may comprise at least one switch configured to generate a control signal. For example, an input device may comprise a touch surface for a user to provide input (e.g., finger contact to the touch surface) corresponding to a control signal. An input device comprising a touch surface may be configured to detect contact and movement on the touch surface using any of a plurality of touch sensitivity technologies including capacitive, resistive, infrared, optical imaging, dispersive signal, acoustic pulse recognition, and surface acoustic wave technologies. In variations of an input device comprising at least one switch, a switch may comprise, for example, at least one of a button (e.g., hard key, soft key), touch surface, keyboard, analog stick (e.g., joystick), directional pad, mouse, trackball, jog dial, step switch, rocker switch, pointer device (e.g., stylus), motion sensor, image sensor, and microphone. A motion sensor may receive user movement data from an optical sensor and classify a user gesture as a control signal. A microphone may receive audio data and recognize a user voice as a control signal.

A haptic device may be incorporated into one or more of the input and output devices to provide additional sensory output (e.g., force feedback) to the user. For example, a haptic device may generate a tactile response (e.g., vibration) to confirm user input to an input device (e.g., touch surface). As another example, haptic feedback may notify that user input is overridden by the computing device.

### Network

In some variations, the systems and methods described herein may be in communication with other computing devices via, for example, one or more networks, each of which may be any type of network (e.g., wired network, wireless network). The communication may or may not be encrypted. A wireless network may refer to any type of digital network that is not connected by cables of any kind. Examples of wireless communication in a wireless network include, but are not limited to cellular, radio, satellite, and microwave communication. However, a wireless network may connect to a wired network in order to interface with the Internet, other carrier voice and data networks, business networks, and personal networks. A wired network is typically carried over copper twisted pair, coaxial cable and/or fiber optic cables. There are many different types of wired networks including wide area networks (WAN), metropolitan area networks (MAN), local area networks (LAN), Internet area networks (IAN), campus area networks (CAN), global area networks (GAN), like the Internet, and virtual private networks (VPN). Hereinafter, network refers to any combination of wireless, wired, public and private data networks that are typically interconnected through the Internet, to provide a unified networking and information access system.

Cellular communication may encompass technologies such as GSM, PCS, CDMA or GPRS, W-CDMA, EDGE or CDMA2000, LTE, WiMAX, and 5G networking standards. Some wireless network deployments combine networks from multiple cellular networks or use a mix of cellular, Wi-Fi, and satellite communication.

### II. Methods

Also described here are methods for evaluating and predicting a risk of a chronic medical condition of a user using the systems and devices described herein. Analysis of user data and sleep measurement data may be used to generate one or more of: insights into user health (e.g., trends or other observations related to sleep, physical health, and/or mental health), a prediction of a risk of a medical condition such as a sleep disorder, and one or more recommendations (e.g., actionable prompt) that may suggest a tangible step that the user take such as making a change in behavior and/or a change in device settings. The insights generated from sleep data may raise awareness that sleep is foundational to overall health and encourage users to continue self-reporting outcomes. The recommendations may in some cases encourage users to take their sleep data to clinical action (e.g., seek a referral to a sleep service).

In some variations, a user may download and execute a mobile application and/or register through a web portal. In some variations, user registration and onboarding may be automated or semi-automated. As part of an onboarding process, user demographics (e.g., age, sex, height, weight), medical history (e.g., comorbidities, symptoms, medication), and/or any suitable user information may be entered in the system and added to a user profile.

During user onboarding, a user may optionally complete an intake questionnaire or other suitable intake form (e.g., dynamic or static questionnaire, such as a self-screening tool). The questionnaire may, for example, be tailored for a sleep-related disorder. For example, a user may be asked to complete a questionnaire relating to personal medical history (e.g., symptoms, diagnosis of a chronic disease, medications), family medical history, and/or current symptoms related to sleep (e.g., insomnia). Furthermore, the questionnaire may additionally or alternatively include generalized questions that are not necessarily medical condition-specific (e.g., sleep habits, sleep goals, mental health goals, physical health goals, nutrition).

Generally, the methods described herein may include performing an objective and subjective assessment of patient sleep, generating sleep insights for the user, and providing sleep recommendations for the user's consideration and action. It should be appreciated that any of systems and devices described herein may be used in the methods described herein.

FIG. 3A is flowchart that generally describes a sleep disorder prediction process (300). The process (300) may include a sleep assessment process (302), a sleep insight process (304), and a sleep recommendation process (306). The sleep assessment process (302) may include outputting a user input notification for user data (e.g., user input of subjective sleep data) using a computing device (310). For example, the computing device may display a prompt at predetermined intervals as a reminder for user input for an assessment (e.g., wake time, bedtime). The notification may be provided periodically, such as at least once daily, twice daily (e.g., morning, evening), thrice daily (e.g., morning, afternoon, evening), and the like. In some variations, the notification can be output at around a predetermined wake time (e.g., about 6 am) and around a predetermined bedtime of a user (e.g., about 10 μm) that may be set to a default or input by a user. Although twice daily notifications and assessments are described herein, any number of notifications and assessments may be performed on a daily basis. For example, an afternoon notification and assessment may be performed to receive user data corresponding to a nap. In some variations, the notification can additionally or alternatively be configured to output at a predetermined time with a predetermined duration, and/or frequency. For example, the notification may include an audible notification (e.g., sound such as musical tone, beep, chirp), visual notification (e.g., flashing lights, graphical display), physical notification (e.g., haptic feedback through a vibratory motor), and any combination thereof. In some variations, if the user fails to respond to the notification (e.g., perform an assessment) within a predetermined period of time after receiving the notification (e.g., 1 minute, 2 minutes, 5 minutes, 10 minutes, etc.), then the notification may be provided again. Alternatively, in some variations, the notification may be omitted.

User data and/or sleep measurement data may be received at a device (320) such as a computing device (110), a measurement device (120), a database (140), a server (150), and an HCP device (160) as described herein. In some variations, the user data may comprise a sleep rating, a physical health rating, and a mental health rating. For example, the sleep rating may comprise a sleep quality status, the physical health rating may comprise a body status, and the mental health rating may comprise an emotional status. Furthermore, the sleep quality status may comprise a wake reason. The body status may comprise a body discomfort type a body discomfort area, and a body discomfort intensity. The emotional status may comprise a wake time emotion and a bedtime emotion. In some variations, the sleep rating, the physical health rating, and the mental health rating may each correspond to a predetermined scale. For example, the predetermined scale may be a Likert scale (e.g., 3 point scale, 5 point scale, 10 point scale, n point scale).

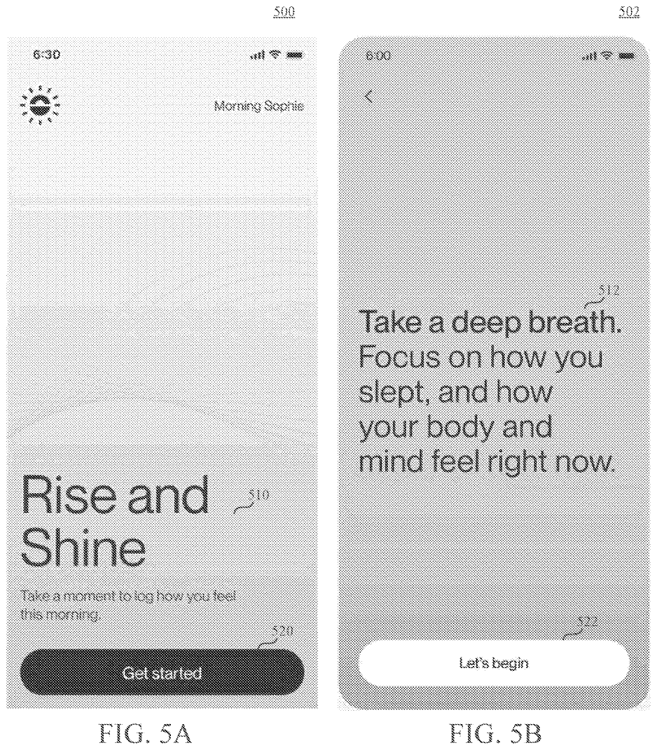

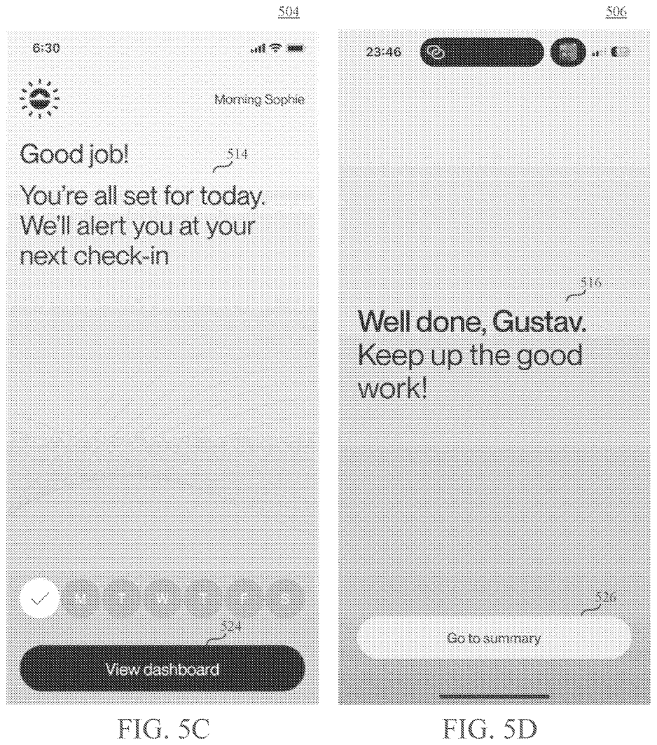

In some variations, the user data may be input on a GUI of the computing device as described in more detail with respect to FIGS. 5A-9B. In some variations, the user data input by a user may be in response to a predetermined set of sleep and/or health-related questions. The questions may be based on a set of clinically recognized sleep questionnaires including, but not limited to, the Functional Outcomes of Sleep Questionnaire (FOSQ), the Idiopathic Hypersomnia Severity Scale (IHSS), the Epworth Sleepiness Scale, the Berlin Questionnaire for Sleep Apnea, the Reduced Morning-ness/Evening-ness Questionnaire (rMEQ), the Insomnia Severity Index, the Sleep Inertia Questionnaire (SIQ), PHQ-9, the American Thoracic Society sleep-related questionnaires, and the like.

In some variations, the questions to be asked may be generated based on a predetermined list of questions. At least a portion of the questions asked to the user may be static or set to be asked by default. Additionally or alternatively, at least a portion of the questions asked to the user may be asked at different frequencies (e.g., daily, weekly, biweekly, monthly). Additionally or alternatively, at least a portion of the questions asked to the user may be dynamic or set to be asked in response to current user data (e.g., user answers to static questions). For example, in response to a prediction of a high risk of a sleep disorder, dynamic questions may be generated to gather further information to help characterize the risk of the sleep disorder. In other words, dynamic questions may include one or more follow-up questions that are customized to correlate to different risk levels. The types of dynamic questions may additionally or alternatively be medical condition-specific and user-specific.

In some variations, one or more of a computing device and a measurement device may be configured to measure sleep measurement data corresponding to one or more sleep parameters of the user. The sleep measurement data may be transmitted to the computing device (or any of the devices described herein) for processing and analysis. For example, a communication channel may be established between the measurement device and a computing device. The communication channel may be a wired or wireless connection and use any communication protocol including but not limited to those described herein. The communication channel may be established at predetermined intervals based on one or more of time (e.g., hourly, daily, weekly, etc.), device usage (e.g., after a wake time, upon device power on), request for connection, and the like.

In some variations, the sleep measurement data may comprise one or more sleep parameters. The one or more sleep parameters may comprise one or more of a sleep start time, a sleep end time, a sleep duration, a time to fall asleep, a number of times woken, a wake period duration, a snore status, a snore duration, an exercise duration, an exercise start time, an exercise end time, a number of alcoholic drinks consumed, a time of last alcoholic drink consumed, a number of caffeine drinks consumed, a time of last caffeine drink consumed, a daytime sleepiness status, a daytime sleepiness severity, a daytime sleepiness status, a daytime sleepiness severity, an average sleeping oxygen saturation, an average sleeping heart rate variability, a minimum sleeping heart rate, a maximum sleeping heart rate, an average sleeping heart rate, a set of medications consumed, and user demographic data. In some variations, a sleep parameter may further comprise a user wake time note and a user bedtime note. In some variations, user demographic data may comprise one or more of medical history and test results.

Additionally, or alternatively, the user data may be transcribed and/or parsed using a suitable voice-to-text transcription model or service (e.g., Google Voice or the like). Optionally, the collection and storage of user data may be performed in a HIPAA-compliant manner. Furthermore, in some variations, some or all user data may be encrypted and/or de-identified to further ensure user privacy. For example, in these variations, the de-identification may provide an additional layer of security in combination with HIPAA-compliant practices to prevent other third parties from having access to identifiable user data.

In some variations, user data and sleep measurement data may be integrated to generate sleep data. For example, sleep data may be generated based on the user data and/or sleep measurement data (330). In some cases, the user data and the sleep measurement data may not match due to error of one or more of a user and a measurement device. For example, a measurement device may measure a wake time as 7:00 am while the user data may reflect a wake time of 6:45 am. Accordingly, a user during a wake or bedtime assessment may reconcile user data and the sleep measurement data by confirming the correct wake time between the two to ensure data integrity and to generate sleep data. As another example, a measured bedtime (e.g., 11:00 μm) (620) in GUI (602) may be pre-populated with sleep measurement data, but may be manually confirmed by a user to reflect an actual bedtime. In some variations, sleep data generation (e.g., data integration, reconciliation) may comprise formatting the data to permit comparison and analysis across sets of data generated from different devices. In some variations, data integration may be performed using, for example, one or more of a computing device, a measurement device, and a server.

In some variations, the sleep assessment (302) may be performed at predetermined times during the day. For example, in some variations, the sleep assessment (302) may be performed twice daily, once in the morning and once in the evening. In some variations, the sleep assessment (302) may include a wake assessment (321) (i.e., at or close in time (e.g., within 30-60 minutes) to a user's wake time) and a bedtime assessment (326). (i.e., at or close in time (e.g., within about 30-60 minutes) to a user's bedtime). FIG. 3B is flowchart that generally describes a wake assessment process (321). The wake assessment process (321) may include outputting a user input notification for user data (e.g., user input of subjective sleep data) using a computing device (322). Sleep measurement data may be received from one or more measurement devices (323) and user data may be received at a computing device (324). Sleep data may be generated based on the user data and/or sleep measurement data (325). In some variations, the user may be prompted at a wake time assessment to input one or more of a prior bedtime (e.g., prior night's bedtime), a time to fall asleep, a wake time, a number of times woken, a wake period duration (e.g., total time awake between bedtime and wake time), wake reason, body status (e.g., how body feels on a scale from 1-5, physical discomforts and intensity), emotional status (e.g., how user feels today on a scale from 1-5, emotional feeling, why user feels that emotion), and a wake time note.

FIG. 3C is flowchart that generally describes a bedtime assessment process (326). The bedtime assessment process (326) may include outputting a user input notification for user data (e.g., user input of subjective sleep data) using a computing device (327). User data associated with sleep may be received at a computing device (328). Sleep data may be generated based on the user data and/or sleep measurement data previously received (329). In some variations, the user may be prompted at a bedtime assessment to input one or more of an emotional status (e.g., how user feels today on a scale from 1-5, emotional feeling, why user feels that emotion), daytime sleepiness status and severity, and factors that may impact sleep (e.g., medication, alcohol, caffeine, exercise, nap frequency and/or nap duration), and a bedtime note.

Turning back to FIG. 3A, As shown there, the sleep insight process (304) may include generating one or more trends and, in some variations, optionally generating a predicted risk of one or more sleep disorders. For example, one or more trends (e.g., sleep trends, health trends) may be generated based on the sleep data (340). As depicted in FIGS. 10A-10G, the sleep data may be analyzed to generate one or more trends that may be output on a GUI. For example, sleep trends may include one or more of sleep quality, average sleep time, average time asleep, average time to fall asleep, average wake time, average snoring time, physical discomforts, sleeping heart rate (e.g., maximum, minimum, and/or average), sleep disruptions, average sleeping oxygen saturation, average heart rate variability, sonar data, and the like. In some variations, a user may select a time period for trend analysis (e.g., one week, two weeks, three weeks, one month, two months, n weeks, n months). In some variations, trend analysis may incorporate the user data (e.g., user perception of body pain levels) and sleep measurement data (e.g., average heart rate, sonar data). Any combination of the user data and sleep measurement data described herein may be used. In some variations, the trends may be output using one or more of plain language (e.g., “Your morning headaches are becoming more frequent.”), graphical visualizations, charts, plots, summaries, and the like. In some variations, one or more of the sleep data, trends, and predictions may be transmitted to a predetermined contact such as a sleep service (e.g., clinician, specialist, doctor, health care professional) and/or a predetermined contact (e.g., family member, friend, caregiver). In some variations, one or more of the GUIs may include one or more selectable section navigation headers (not shown) to facilitate navigation to desired trends, data, and predictions.

In some variations, a risk of a sleep disorder may be predicted based on the sleep data (350). For example, a prediction step may include performing analytics to assess the sleep data for prediction of a risk of one or more sleep disorders. In some variations, the sleep data may be analyzed via machine learning and/or other artificial intelligence techniques, and an output may be created as a prediction of user risk of a sleep disorder (e.g., predicted severity, predicted likelihood of a medical condition such as sleep apnea). In some variations, the sleep data across multiple users may be analyzed in order to characterize each user's risk of a sleep disorder or other condition. In some variations, suitable alert conditions may be selected, such as thresholds and/or other conditions for triggering a notification associated with a high-risk user.

In some variations, methods, systems, and apparatus, for predicting a risk of a sleep disorder may utilize one or more predictive models (e.g., machine learning models) such as logistic regression, decision trees, and neural networks. For example, a system may receive sleep data (e.g., as described herein) from a plurality of users, process the data into an encoded representation and generate one or more predictions on the data. A neural network may be trained to process sleep data using a recurrent neural network to predict a risk of one or more sleep disorders. New data collected over time may improve and refine the predictive model(s). In some variations, a dimensionality of the sleep data may be reduced (e.g., using Principal Component Analysis) to discover correlations between the features. For example, principle components can be accepted up to the point that 90% of the variance of the data is explained with the principle component mappings. Due to the nature of sleep data collection (e.g., forgetfulness in performing assessments), some users may miss inputting at least some user data for an expected assessment. To compensate for missing user data, in some variations, the missing values, may be set as being the previous input values or, a baseline average for the user or users in a same demographic group as the user. In some variations, the recurrent neural network model may be trained to predict the next day's assessment values, so that in the event of missing data, the recurrent neural network can fill in the missing user data with predictions.

In some variations, a recurrent neural network may take as inputs sleep data and outputs the risk (e.g., probability) of a user having one or more sleep disorders. In some variations, the one or more sleep disorders may comprise obstructive sleep apnea, central sleep apnea, hypoapnea, orthopnea, nocturnal atrial fibrillation, nocturnal hypertension, nocturnal discomfort, evening exacerbated chronic obstructive pulmonary disease, heart failure, asthma, sleep quality, and waking discomfort. In some variations, after a period of model training with an initial dataset, the model may be deployed to predict the probability that user has one or more sleep disorders. In some variations, results of the prediction using the model may be used to generate one or more of a notification, an actionable suggestion, and a referral.

To generate suitable training data for training the machine learning model, one or more extracted features may be derived from suitable training datasets. Generally, the training datasets may include information relating to user data, sleep measurement data, and clinical diagnosis, and any such information may be extracted as features for training a machine learning model. Any suitable piece of information from the training datasets (e.g., any information from the training datasets described above) may be extracted as a feature and/or standardized in format for use in training the machine learning model. In some variations, some extracted features may be combined or otherwise correlated to one another for training.

In some variations, the trained machine learning model(s) may be tested to evaluate the performance of the trained model(s) on another set of sleep data (e.g., consumer data, sample data). The model may thereafter be modified to improve performance. Further validation of the model may be performed using an independent set of sleep data to confirm effectiveness. Once deployed, the model may be configured to generate predictions in real-time (e.g., via a web or mobile application on a computing device). The deployed model's performance may be monitored over time to identify potential issues and to improve accuracy. The model may be periodically retrained with different sleep data to improve performance and accuracy over time.

Referring back to FIG. 3A, optionally, in some variations, methods may include generating a GUI including one or more of the trend and risk of sleep disorder (360). As described in more detail with respect to FIGS. 10A-10G, 11A, 11B, 13, 14A, and 14B, the trends and predictions may provide user insight into one or more of their sleep, physical health, and mental health.

In some variations, a sleep recommendation process (306) may include providing one or more of an actionable suggestion and a clinical referral to the user based on the sleep insights. In some variations, an actionable suggestion may be provided to the user (e.g., via the computing device) based on the generated trend and predicted risk of sleep disorder (370). In some variations, an actionable suggestion may comprise modifying computing device settings based on one or more of the user data, sleep measurement data, sleep data, and the user health graphic. For example, a microphone of a measurement device may be configured to record audio of snoring of the user during sleep in response to user selection of a corresponding actionable suggestion (e.g., notification to reduce alcohol consumption).

Optionally, in some variations, a clinical referral may be provided to the user (e.g., via the computing device) based on the trend, risk of sleep disorder, and/or user health graphic (460). In some variations, a clinical referral may include a sleep service (380). For example, a user may be prompted to share a sleep and health report (e.g., GUI (1300)) generated by the computing device with their doctor.

Consistent user engagement with the systems, devices, and methods described herein may improve the accuracy and effectiveness of the generated insights and predictions. However, some users may tend to be discouraged from consistently engaging with the system (e.g., due to stress and/or poor health, or otherwise). Accordingly, in some variations, methods for remotely evaluating and predicting a sleep-related disorder may benefit from display of graphical visualizations configured to improve user engagement and morale. Additionally or alternatively, pleasant (e.g., relaxing, soothing) content may be provided to users who successfully complete daily assessments. For example, methods may include providing pleasant audio and/or visual feedback (e.g., music clips, encouragement).

FIG. 4 is flowchart that generally describes a graphical user health status process (400). The process (400) may include outputting a user input notification for user data using a computing device (410). For example, the computing device may display a prompt at predetermined intervals as a reminder for user input for an assessment (e.g., wake time, bedtime). User data and/or sleep measurement data may be received at a device (420) such as a computing device (110), a measurement device (120), a database (140), a server (150), and an HCP device (160) as described herein. In some variations, the user data may comprise a sleep rating, a physical health rating, and a mental health rating as described herein. For example, the sleep rating, the physical health rating, and the mental health rating may each correspond to a predetermined scale. For example, the predetermined scale may be a Likert scale (e.g., 3 point scale, 5 point scale, 10 point scale, n point scale).

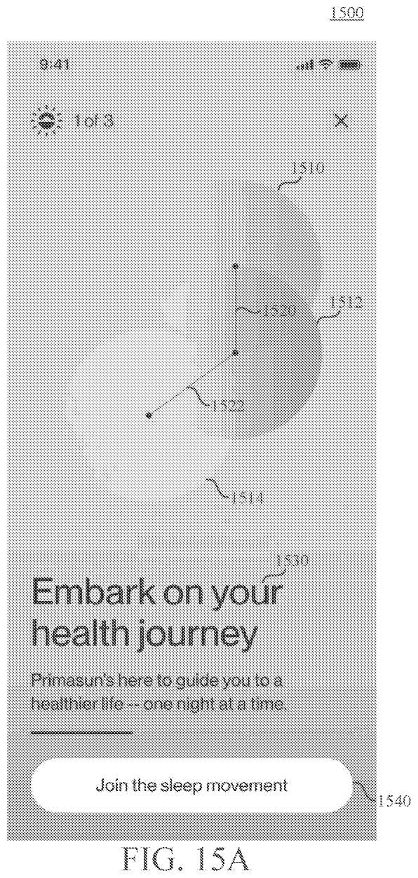

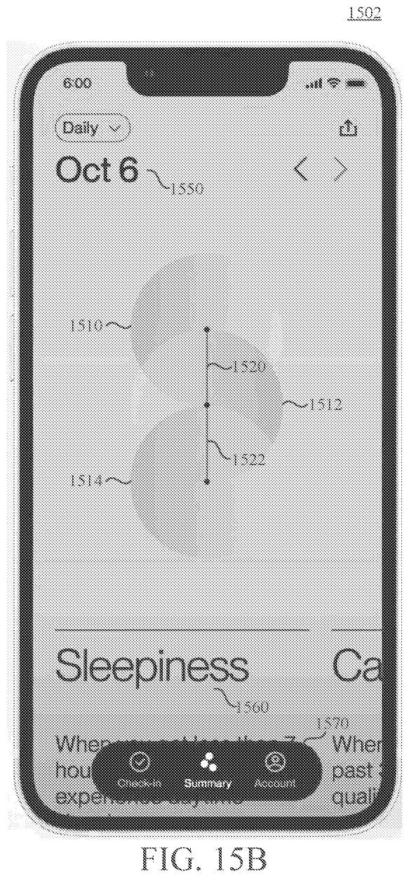

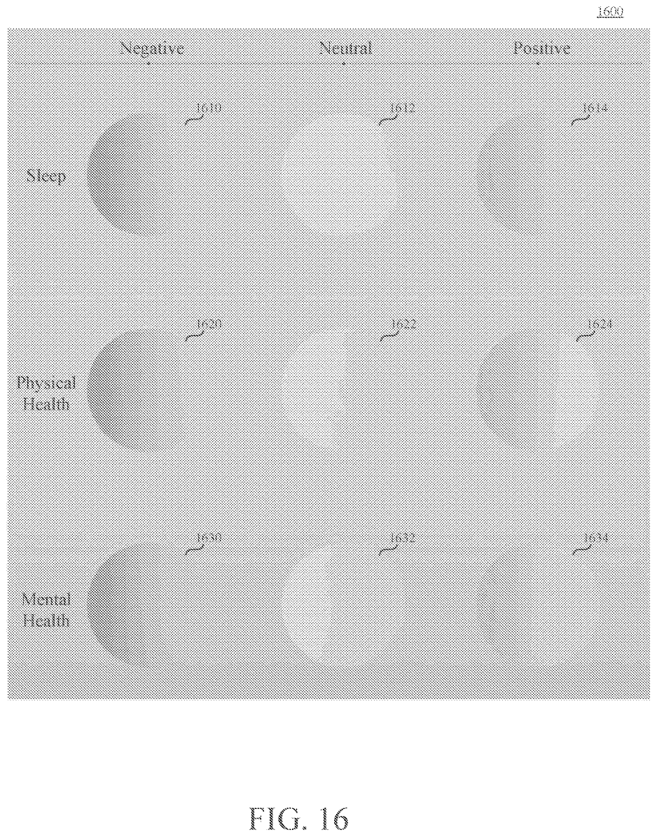

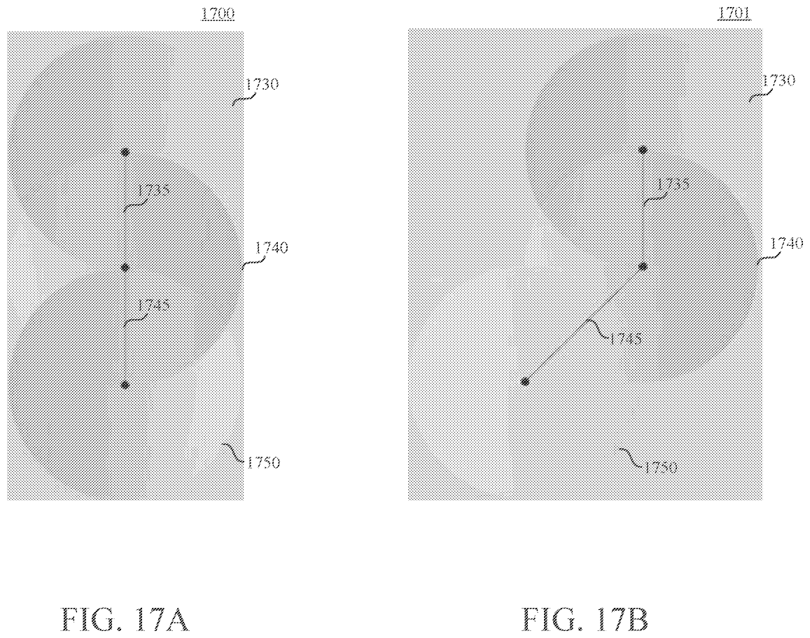

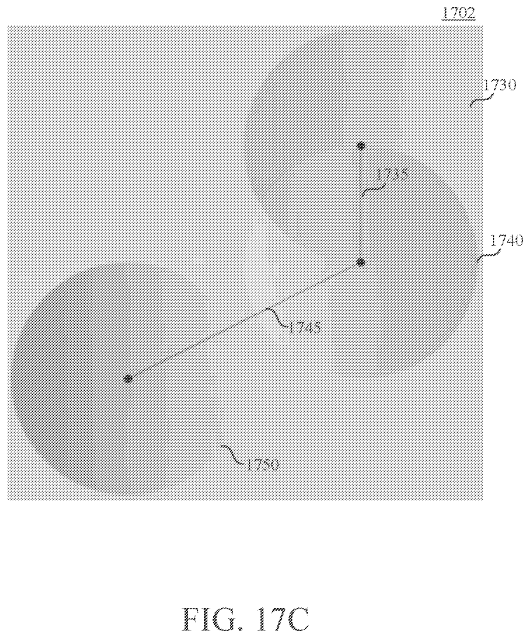

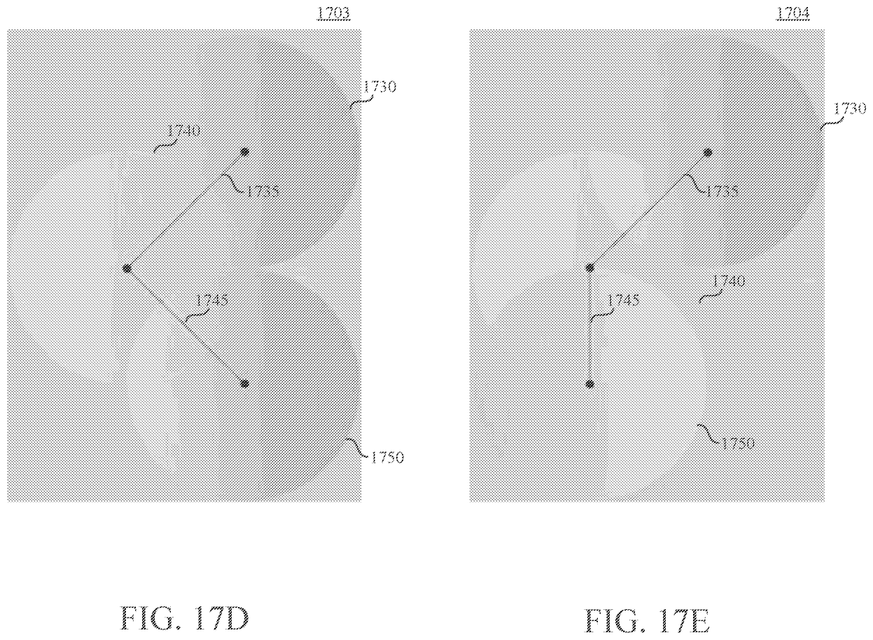

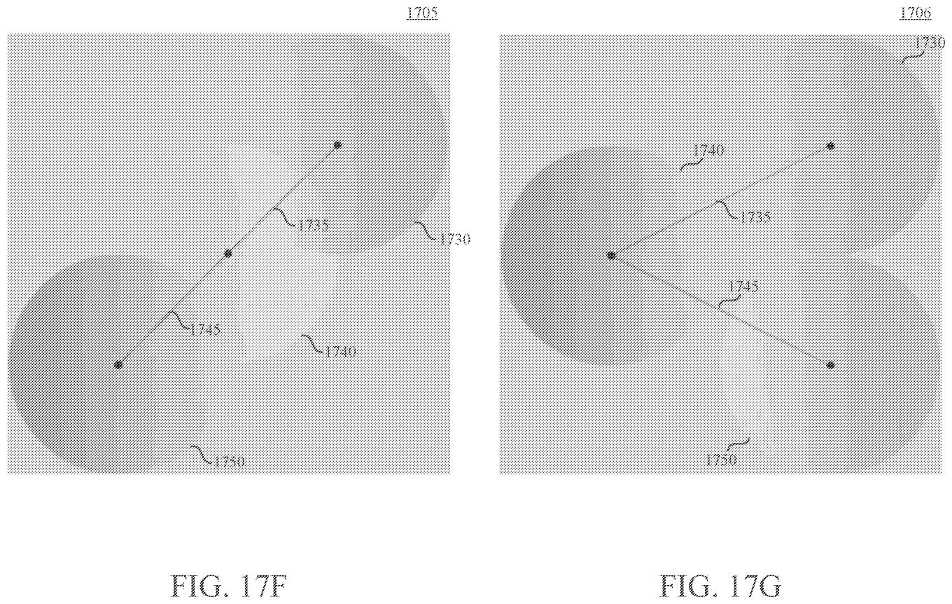

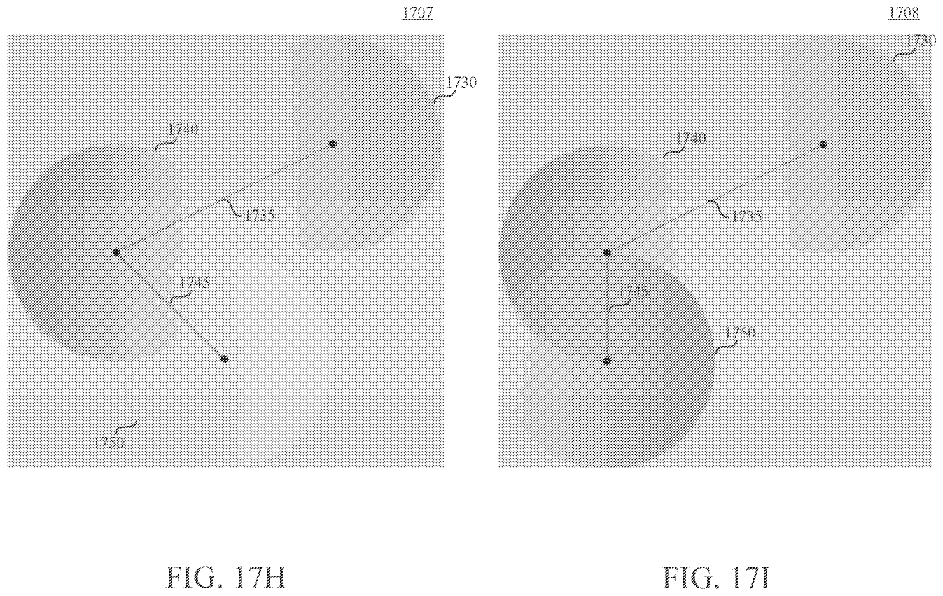

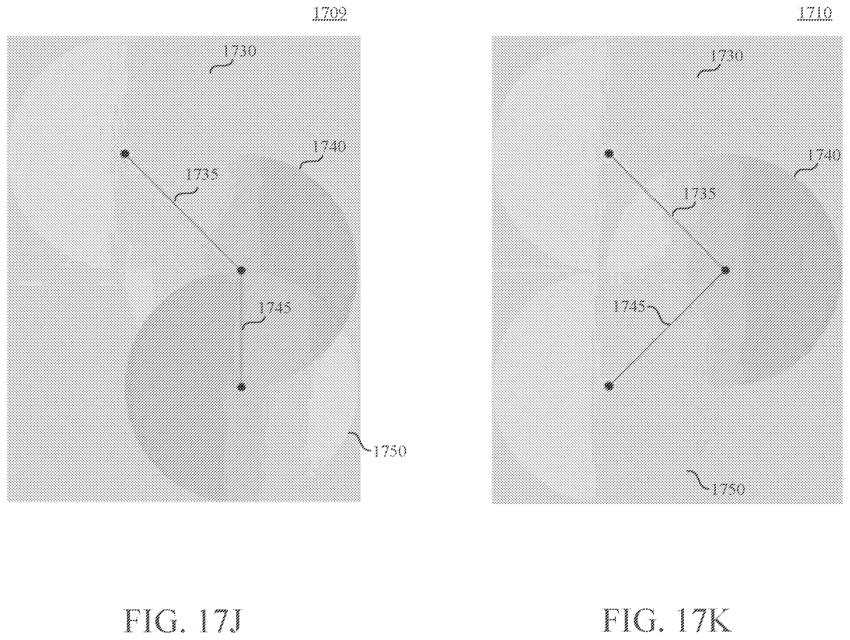

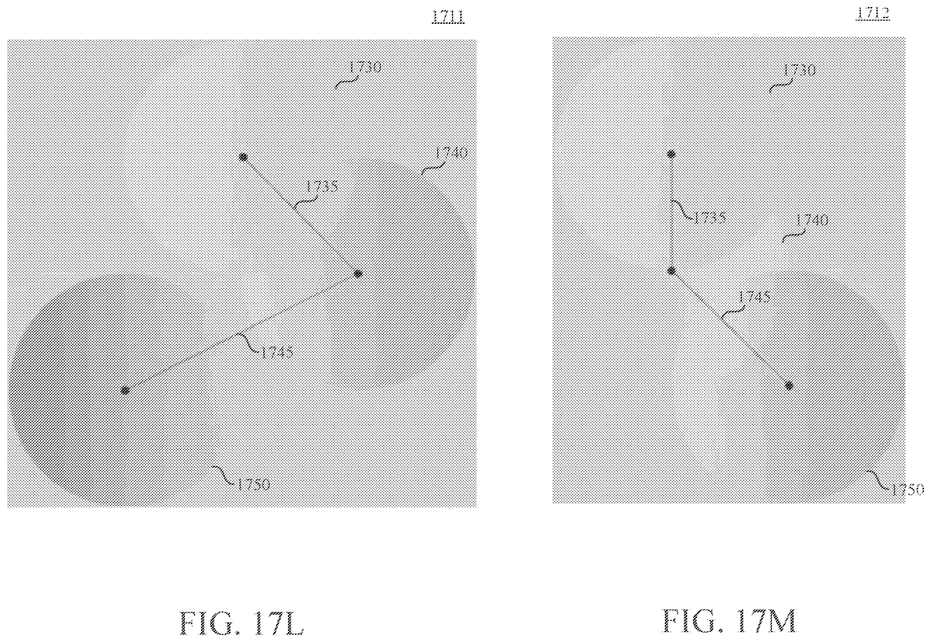

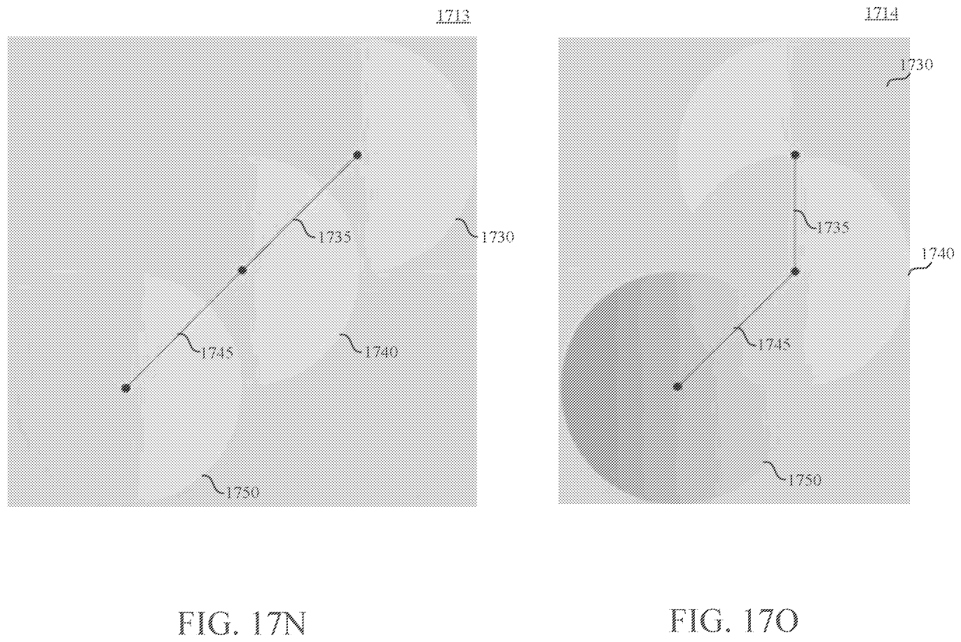

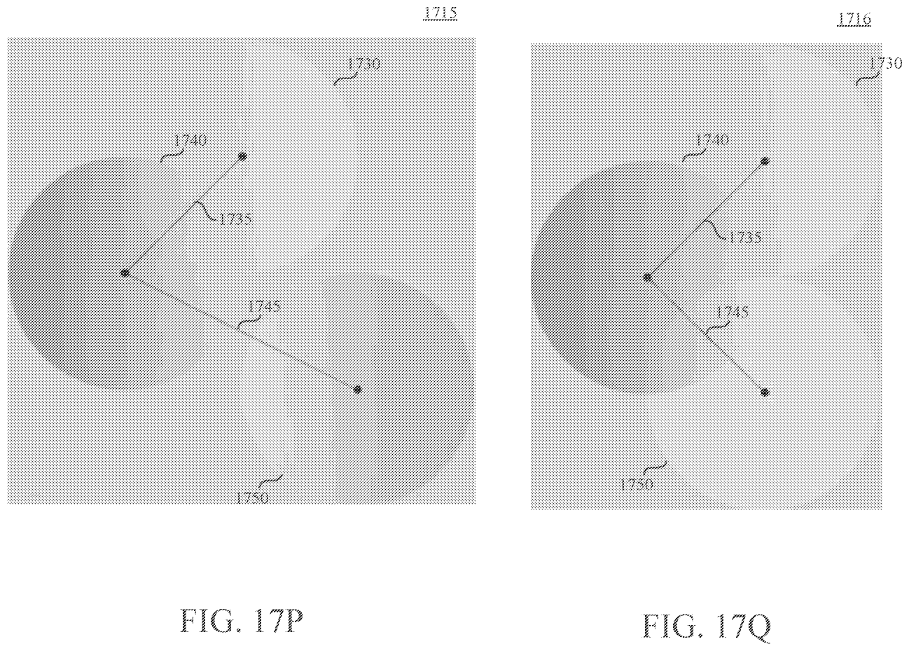

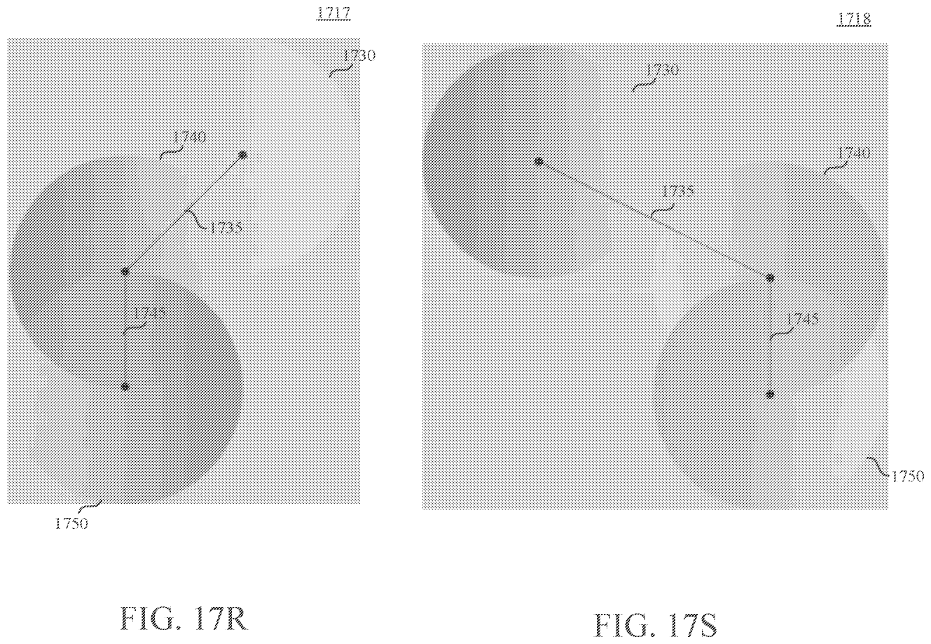

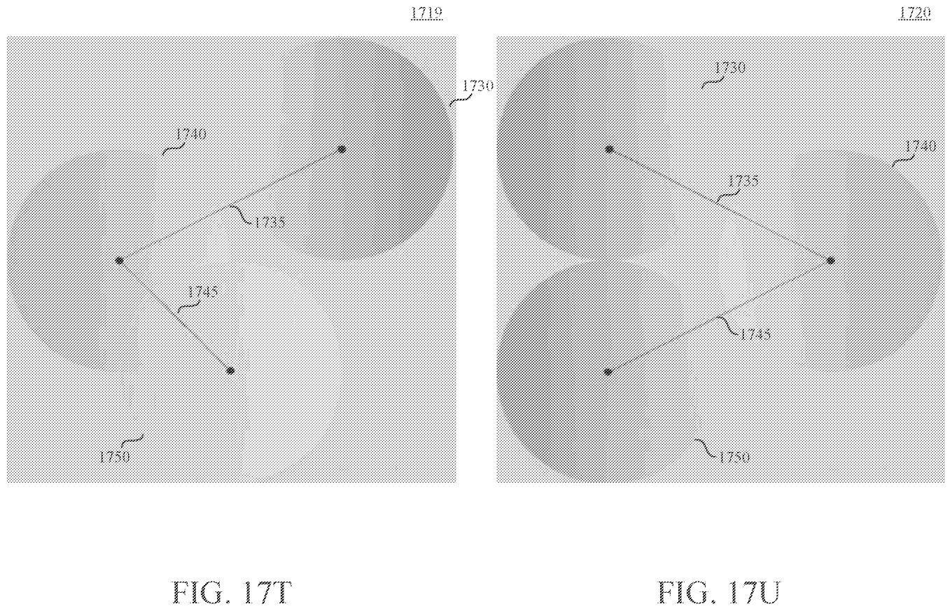

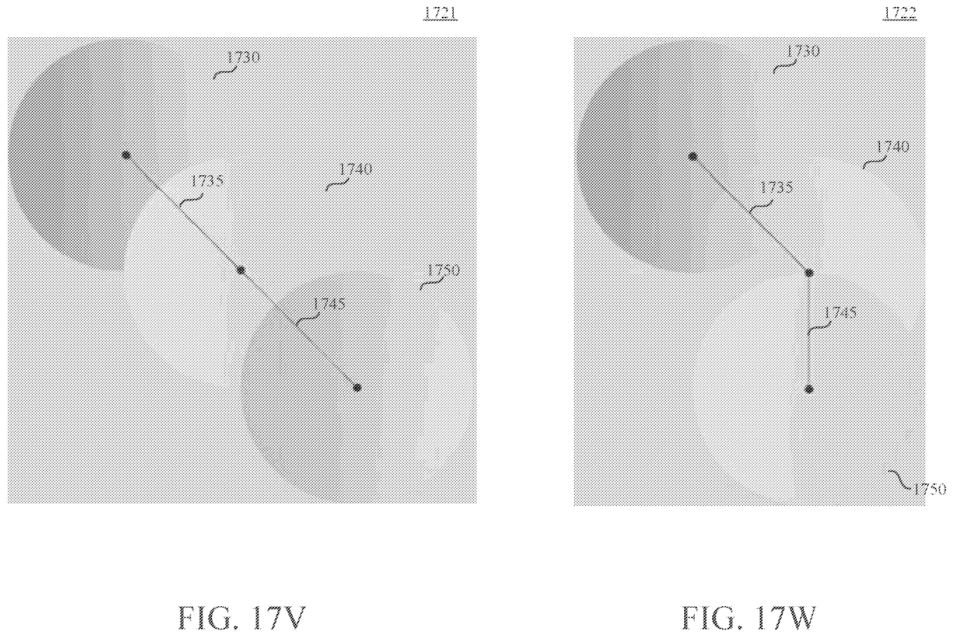

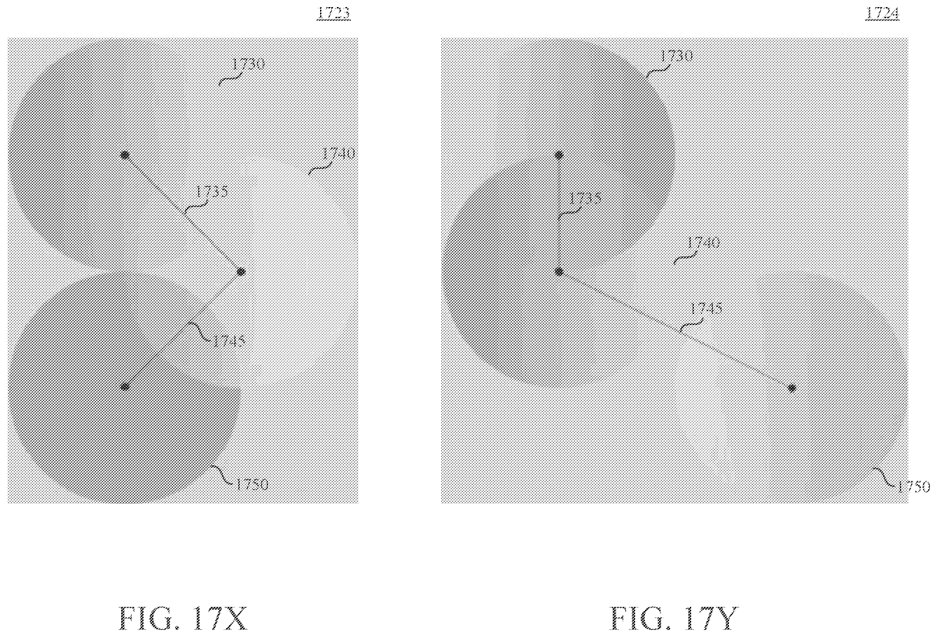

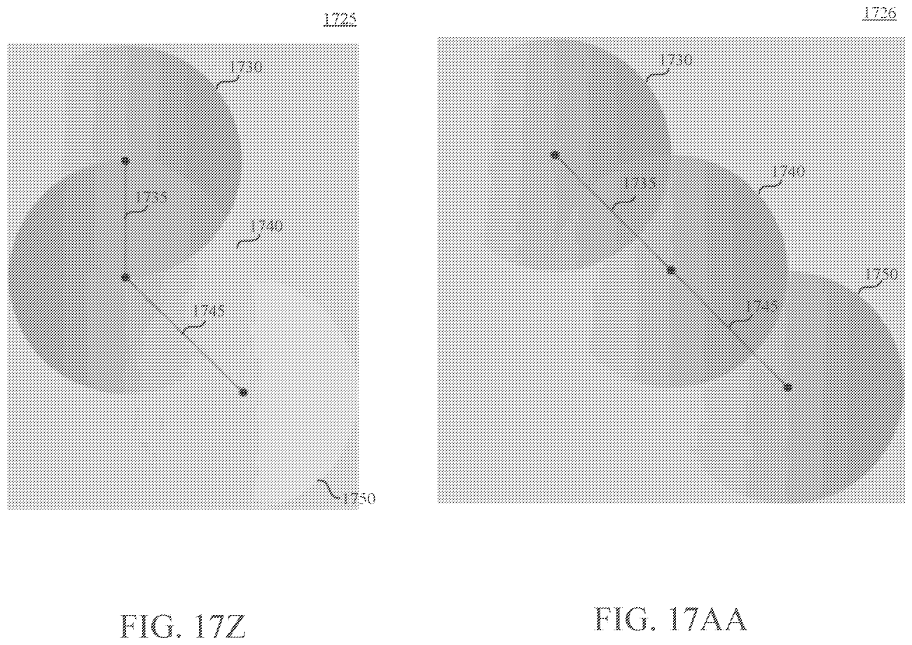

As shown in FIGS. 15A, 15B, and 17A-17AA, the process (400) may include generating a user health graphic on a graphical user interface of the computing device (430). In some variations, the user health graphic may be generated by generating a first shape based on the sleep rating, a second shape based on the physical health rating, and a third shape based on the mental health rating. Additionally or alternatively, the user health graphic may be generated by generating a first color based on the sleep rating, a second color based on the physical health rating, and a third color based on the mental health rating. The first shape may be associated with the first color, the second shape may be associated with the second color, and the third shape may be associated with the third color. For example, FIG. 16 is a schematic diagram of a set of shapes having colors corresponding to different sleep and health ratings. For example, a negative (e.g., subpar) sleep rating may correspond to a first sleep shape (1610) having a first color (e.g., red), a neutral (e.g., moderate) sleep rating may correspond to a second sleep shape (1612) having a second, different color (e.g., yellow), and a positive (e.g., good) sleep rating may correspond to a third sleep shape (1614) having a third, color different from the first and second colors (e.g., green). In some variations, a sleep quality of 1 or 2 on a 5-point Likert scale may correspond to a negative sleep rating, a sleep quality of 3 may correspond to a neutral rating, and a sleep quality of 4 or 5 may correspond to a positive sleep rating.

As discussed herein, a sleep rating and a physical health rating, and a mental health rating each correspond to user input of user data. In some variations, a sleep quality rating may be based on at least the sleep rating and may include additional sleep parameters as discussed herein. Similarly, a physical health quality rating may be based on at least the physical health rating and may include additional physical health parameters as discussed herein.

In some variations, a negative sleep quality rating may be below about 75% (e.g., out of a 100% scale), below about 70%, below about 65%, or below about 60%, including all sub-ranges and values in-between. In some variations, a neutral sleep quality rating may be between about 60% and about 85%, between about 65% and about 80%, between about 70% and about 80%, between about 75% and about 80%, or between about 60% and about 80%, including all sub-ranges and values in-between. In some variations, a positive sleep quality rating may be above about 70%, above about 75%, above about 80%, or above about 85%, including all sub-ranges and values in-between. A sleep quality rating may be determined based on a plurality of subjective and/or objective sleep parameters such as a sleep rating, a sleep duration, sleeping heart rate variability, and the like.

In some variations, a sleep quality rating may be determined based on a plurality of subjective and/or objective sleep parameters such as a sleep rating (e.g., user input of subjective sleep quality), a sleep duration, sleep efficiency, sleep latency, number of awakenings (e.g., awakenings lasting more than a predetermined threshold (e.g., 4 minutes, 5 minutes, 6 minutes, 7 minutes, or longer), time spent awake (e.g., wake after sleep onset (WASO)), sleeping heart rate variability, sleep time consistency (e.g., consistency of a fall asleep time), sleep duration consistency (e.g., consistency of time slept), check-in consistency (e.g., user data input consistency), and the like. In some variations, the number of sleep parameters used to determine the sleep quality rating may change over time as data is collected. For example, sleep time consistency may be determined using a plurality of sleep times over a plurality of nights and may be a sleep parameter added to a determination of the sleep quality rating after a predetermined number of nights (e.g., 3 nights, 5 nights, 7 nights, 14 nights).

In some variations, the sleep quality rating may be based on a weighted combination of the plurality of subjective and/or objective sleep parameters. For example, the sleep quality rating may be based on a weighted combination of two or more of: sleep duration, sleep efficiency, sleep latency, number of awakenings, time spent awake, and sleep rating. In some variations, each sleep parameter may be weighted the same or differently. For example, each sleep parameter of sleep duration, sleep efficiency, sleep latency, number of awakenings, time spent awake, sleeping heart rate variability, etc. may be given equal weight while sleep rating may be given a higher relative weight. As discussed above, the sleep rating may correspond to a predetermined scale such as a Likert scale (e.g., five point scale).

An exemplary sleep quality rating may be determined as shown in Table 1 where user data and sleep measurement data correspond to predetermined sleep rating values and are weighted to determine a sleep quality rating.

TABLE 1

| Sleep Parameter | Sleep Quality Value |
|---|---|
| Sleep duration | First age range |
| | xₐ-x_{b} time range = maximum points |
| | x_{c}-x_{d} time range = intermediate points |
| | xₑ-x_{f} time range = minimum points |
| | Second age range |
| | x_{g}-xₕ time range = maximum points |
| | xᵢ-xⱼ time range = intermediate points |
| | xₖ-xₗ time range = minimum points |
| | Third age range |
| | xₘ-xₙ time range = maximum points |
| | xₒ-xₚ time range = intermediate points |
| | x_{q}-xᵣ time range = minimum points |
| Sleep efficiency | First age range |
| | yₐ-y_{b} percentage range = maximum points |
| | y_{c}-y_{d} percentage range = intermediate points |
| | yₑ-y_{f} percentage range = minimum points |
| | Second age range |
| | y_{g}-yₕ percentage range = maximum points |
| | yᵢ-yⱼ percentage range = intermediate points |
| | yₖ-yₗ percentage range = minimum points |
| Sleep latency | xₛ-xₜ time range = maximum points |
| | xᵤ-xᵥ time range = intermediate points |
| | x_{w}-xₓ time range = minimum points |
| Number of awakenings | zₐ-z_{b} range = maximum points |
| | z_{c}-z_{d} range = intermediate points |
| | zₑ-z_{f} range = minimum points |
| Time spent awake | x_{y}-x_{z} time range = maximum points |
| | xₐₐ-x_{bb} time range = intermediate points |
| | x_{cc}-x_{dd} time range = minimum points |
| Sleep quality | aₐ = minimum points |
| | a_{b} = first intermediate points |
| | a_{c} = second intermediate points |
| | a_{d} = third intermediate points |
| | aₑ = maximum points |

In some variations, the time ranges x, percentage ranges y, number ranges z, ratings a, and point value ranges (e.g., minimum, intermediate, maximum) may be predetermined. For example, sleep duration may have a sleep quality point value based on threshold sleep durations (e.g., under 7 hours, between 7 and 9 hours, more than 9 hours) for different age ranges (e.g., young adult, adult, elderly). However, any sleep parameter may be sub-grouped based on age or other demographic category. Sleep efficiency may have a sleep quality point value based on predetermined scale (e.g., 0%-100%) for different age ranges that may be the same or different than the age ranges for sleep duration. Sleep latency and time spent awake (e.g., wake after sleep onset (WASO)) may each have sleep quality point values based on predetermined time thresholds (e.g., up to 30 minutes, between 30-45 minutes, etc.). The number of awakenings may have sleep quality point values based on predetermined ranges (e.g., 0-1, 2-3, etc.). Sleep quality may be subjectively rated on a Likert scale of 1-5 and have corresponding point values.

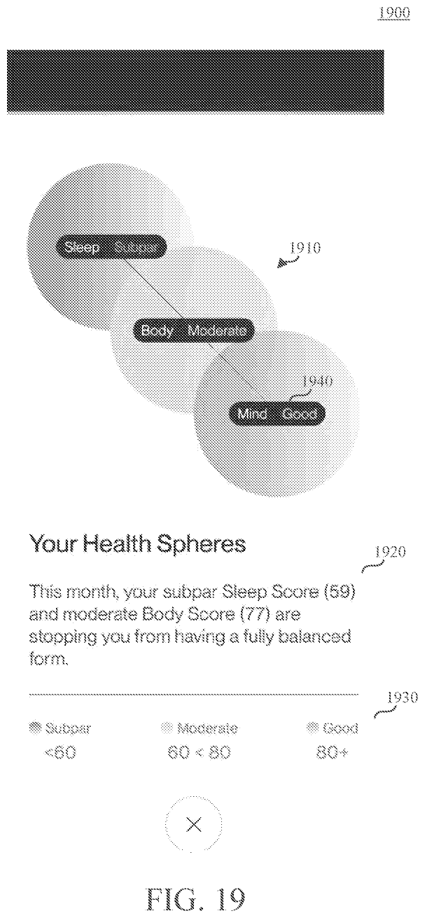

In some variations, a sleep quality rating may be the sum of the sleep quality values of each sleep parameter such that the sleep quality rating may be between a minimum and maximum number of points. In some variations, the sleep quality rating may be scaled such that the maximum number of points corresponds to a sleep quality rating of 100. For example, as shown in FIG. 19, a subpar health quality rating (e.g., subpar sleep quality rating) may be less than about 60 (e.g., less than about 60%), a moderate health rating (e.g., moderate physical health quality rating) may be between about 60 and about 80 (e.g., between about 60% and about 80%), and a good health rating (e.g., mental health quality rating) may be above about 80 (e.g., above about 80%).

In some variations, a sleep quality rating may be determined periodically or at predetermined interval (e.g., periodically, twice daily, daily, twice weekly, weekly, twice monthly, monthly). In some variations, a sleep quality rating may be the same or differently. For example, a weekly sleep quality rating may include an average of the last week's (e.g., last 7 days, last calendar week) daily sleep quality ratings while a monthly sleep quality rating may include a weighted average of the last month's (e.g., last 4 weeks, from the first of the calendar month) weekly sleep quality ratings (e.g., where more recent weeks are given more weight than more past weeks).

In some variations, a physical health quality rating may be determined based on a plurality of subjective and/or objective physical health parameters such as a physical health rating (e.g., user input of subjective physical health), number of physical discomforts, number of maximum value physical discomforts (e.g., maximum discomfort as input by a user, 5 out of 5), average severity of physical discomfort, and the like. In some variations, the number of physical health parameters used to determine the physical health quality rating may change over time as data is collected.

In some variations, each physical health parameter may be weighted the same or differently. For example, if no discomforts are reported, then the physical health quality rating may correspond to just the physical health rating, or if no maximum value physical discomforts are report, the physical health quality rating may correspond to the physical health rating, number of physical discomforts, and average severity of physical discomfort. As discussed above, the physical health parameters may correspond to a predetermined scale such as a Likert scale (e.g., five point scale).

A physical health quality rating may be inaccurately elevated if discomforts are not accurately inputted by the user. In some variations, a user may receive one or more additional prompts to input physical discomforts if no physical discomforts are inputted for a predetermined amount of time (e.g., 7 days, “For more personalized insights on how your body is doing, be sure to track your specific discomforts in the morning check-in”).

An exemplary physical health quality rating may be determined as shown in Table 2 where user data corresponds to predetermined physical health rating values and are weighted equally to determine a physical health quality rating.

TABLE 2

| Physical Health Parameter | Physical Health Quality Value |
|---|---|
| Physical health rating | aₐ (minimum) = minimum points |
| | a_{b} = first intermediate points |
| | a_{c} = second intermediate points |
| | a_{d} = third intermediate points |
| | aₑ (maximum) = maximum points |
| Number of physical | xₐ-x_{b} range = maximum points |
| discomforts | x_{c}-x_{d} range = first intermediate points |
| | xₑ-x_{f} range = second intermediate points |
| | x_{g}-xₕ range = third intermediate points |
| | xᵢ-xⱼ range = minimum points |
| Number of maximum value | yₐ-y_{b} range = maximum points |
| physical discomforts | y_{c}-y_{d} range = first intermediate points |
| | yₑ-y_{f} range = second intermediate points |
| | y_{g}-yₕ range = third intermediate points |
| | yᵢ-yⱼ range = minimum points |
| Average severity of | zₐ-z_{b} range = maximum points |
| physical discomfort | z_{c}-z_{d} range = first intermediate points |
| | zₑ-z_{f} range = second intermediate points |
| | z_{g}-zₕ range = third intermediate points |
| | zᵢ-zⱼ range = minimum points |

In some variations, the ratings a, number ranges x, y, z, and point value ranges (e.g., minimum, intermediate, maximum) may be predetermined. For example, physical health rating may be subjectively rated on a Likert scale of 1-5 and have corresponding point values. Number of physical discomforts, number of maximum value physical discomforts, and average severity of physical discomfort may have respective physical health quality point values based on predetermined number ranges (e.g., 0, 1, 2, 3, 4, 5, 6 or more, 0-2, 3-5, etc.) for different age ranges (e.g., young adult, adult, elderly).

In some variations, a physical health quality rating may be the sum of the physical health quality values of each physical health parameter such that the physical health quality rating may be between a minimum number and a maximum number of points. In some variations, the physical health quality rating may be scaled such that the maximum possible predetermined points corresponds to a physical health quality rating of 100.

In some variations, a physical health quality rating may be determined at a predetermined interval (e.g., periodically, twice daily, daily, twice weekly, weekly, twice monthly, monthly). In some variations, a physical health quality rating may be weighted the same or differently. For example, a weekly physical health quality rating may include an average of the last week's (e.g., last 7 days, last calendar week) daily physical health quality ratings while a monthly physical health quality rating may include a weighted average of the last month's (e.g., last 4 weeks, from the first of the calendar month) weekly physical health quality ratings (e.g., where more recent weeks are given more weight than more past weeks).

In some variations, one or more of the sleep quality rating, physical health quality rating, and mental health quality rating may be output to a user independently of or in conjunction with the user health graphics described herein. Additionally or alternatively, one or more of the sleep quality rating, physical health quality rating, and mental health quality may be used to generate one or more of the shapes and colors of a user health graphic.

In some variations, a negative physical health (e.g., body) rating may correspond to a first physical health shape (1620) having a first color (e.g., red), a neutral physical health rating may correspond to a second physical health shape (1622) having a second, different color (e.g., yellow), and a positive sleep rating may correspond to a third physical health shape (1624) having a third color different from the first and second colors (e.g., green). In some variations, a physical health rating of 1 or 2 on a 5-point Likert scale may correspond to a negative physical health rating, a physical health rating of 3 may correspond to a neutral rating, and a physical health rating of 4 or 5 may correspond to a positive physical health rating.

In some variations, a negative mental health (e.g., mind) rating may correspond to first mental health shape (1630) having a first color (e.g., red), a neutral mental health rating may correspond to a second mental health shape (1632) having a second, different color (e.g., yellow), and a positive sleep rating may correspond to a third mental health shape (1634) having a third color different from the first and second colors (e.g., green). In some variations, a mental health rating of 1 or 2 on a 5-point Likert scale may correspond to a negative mental health rating, a mental health rating of 3 may correspond to a neutral rating, and a mental health rating of 4 or 5 may correspond to a positive mental health rating. In some variations, the predetermined scale may be an n-point scale.

It should be appreciated that while described above as have the same colors denote the same levels (e.g., negative, neutral, positive) across the sleep, physical health, and mental health ratings, this need not be the case and each of the sleep, physical health, and mental health ratings may have their own color schemes denoting the different levels.

In some variations, an arrangement of the first shape, the second shape, and the third shape of a user health graphic on a graphical user interface may be based on the respective sleep and health ratings. For example, FIGS. 17A-17AA depict different color and spatial arrangements of the first shape (1730), the second shape (1740), and the third shape (1750).

In some variations, each of the first shape (1730), the second shape (1740), and the third shape (1750) comprise a two-dimensional geometry. For example, the shapes may be any two-dimensional shape such as a circle, ellipsoid, square, polygon, combinations thereof, and the like. However, in some variations, one or more of the shapes may comprise a three-dimensional geometry. For example, a three-dimensional geometry may include a sphere, cylinder, cone, cube, cuboid, polyhedron, torus, pyramid, prism, combinations thereof, and the like.

For example, a location of the first shape, the second shape, and the third shape may be arranged relative to a first axis of the user health graphic based on the sleep rating, the physical health rating, and the mental health rating. In some variations, the first axis may be a longitudinal axis (e.g., Y-axis) of the user health graphic. In some variations, the longitudinal axis of the user health graphic may be a vertical axis of the display while in other variations it may be a horizontal axis (e.g., X-axis) of the display. The first shape may comprise a first center, the second shape may comprise a second center, and the third shape may comprise a third center. In some variations, a center-to-center distance between the first shape and the second shape may be based on a differential between two of the sleep rating, the physical health rating, and the mental health rating. For example, in FIG. 17H, a center-to-center distance between the first shape (1730) and the second shape (1740) is based on a differential between a rating of the first shape and a rating of the second shape. That is, the relatively longer center-to-center distance between the first shape (1730) and the second shape (1740) is reflected in the larger differential between the positive rating of the first shape (1730) (e.g., green color) and the negative rating of the second shape (1740) (e.g., red color) in comparison to the distances and ratings between the first shape (1730) and the third shape (1750).

In the variation shown in FIG. 17A, the first center, the second center, and the third center are positioned on a first axis. Also shown in FIG. 17A, the centers of two or more of the first shape, the second shape, and the third shape are spaced apart from each other along the first axis. Furthermore, the first shape (1730), the second shape (1740), and the third shape (1750) partially overlap along the first axis. In FIG. 17A, the first center, the second center, and the third center are spaced apart by an equal center-to-center distance along the first axis. Moreover, in the variation shown in FIG. 17A, all three of shapes have the same color, which may indicate that all three ratings are within the same range (e.g., all positive). Furthermore, the vertical and linear alignment of the three shapes indicates that the three ratings are within the same range.

In the variations shown in FIGS. 17B and 17C, the first center and the second center of the first and second shapes (1730, 1740) are positioned on the first axis, and the third center of the third shape (1750) is offset from the first axis (e.g., laterally offset from the first axis). For example, the first shape (1730) and the second shape (1740) having a green color and a corresponding positive rating are positioned on the first axis while the third shape (1750) in FIG. 17B having a yellow color and a neutral rating is laterally offset with respect to the first axis. Similarly, the third shape (1750) in FIG. 17C having a red color and a negative rating is laterally offset with respect to the first axis. The differential in the rating of the shapes may determine a degree of offset between the shapes. For example, a center-to-center distance between the second shape (1740) and the third shape (1750) is greater in FIG. 17C than in FIG. 17B.

In some variations, one of the first center, the second center, and the third center are positioned on the first axis, and the other two of the first center, the second center, and the third center are offset from and on the same or opposing sides of the first axis. In the variations shown in FIGS. 17F, 17N, 17V, and 17AA, the first axis is an angled axis between a longitudinal axis of the user health graphic and a lateral axis of the user health graphic. For example, the angled axis is shown as about 45 degrees but may be any angle between the longitudinal axis and the lateral axis (e.g., about 15 degrees, about 30 degrees, about 45 degrees, about 60 degrees, about 75 degrees).

As shown in FIGS. 17F, 17H, 17L, 17P, 17T, 17V, and 17AA, the centers of two or more of the first shape, the second shape, and the third shape are offset from each other relative to the first axis and/or the first center, the second center, and the third center are laterally offset from each other. For example, each of the first, second, and third shapes may have a different color/rating.

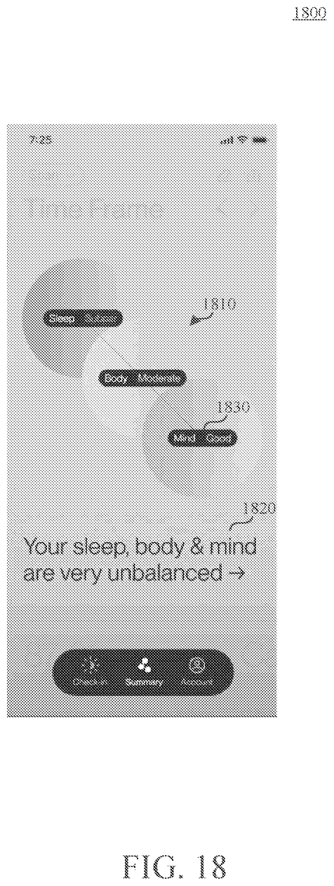

FIGS. 18 and 19 are a set of illustrative variations of a health status graphic and sleep insight graphical user interface configured to facilitate an intuitive and quick understanding of a user's holistic health (e.g., sleep, physical health, mental health) with a combination of graphic and text forms.

FIG. 18 depicts a sixty-second GUI (1800) including a health status graphic (1810) and an insight (1820) providing a text summary corresponding to the health status graphic (1810) for a predetermined time period. In some variations, the health status graphic (1810) may include an overlay of text (1830) indicating the parameter (e.g., sleep, physical health, mental health) and rating (e.g., subpar, moderate, good).

FIG. 19 depicts a sixty-third GUI (1900) including a health status graphic (1910) and an insight (1920) for a predetermined time period (e.g., month) providing a plain language text summary corresponding to the health status graphic (1910). In some variations, the health status graphic (1910) may include an overlay of text (1940) indicating the parameter (e.g., sleep, physical health, mental health) and rating (e.g., subpar, moderate, good). In some variations, the GUI 1900 may include a set of rating thresholds (1930) corresponding to a health quality rating (e.g., sleep quality rating, physical health quality rating, mental health quality rating).

Optionally, in some variations, an animation of a plurality of user health graphics may be generated (440). For example, an updated user health graphic and one or more previous user health graphics corresponding to a predetermined period of time (e.g., one week, two weeks, three weeks, one month) may be displayed sequentially in an animation for a user to gauge their health status over time.

Optionally, in some variations, a process (400) may include providing one or more of an actionable suggestion (450) and a clinical referral (460) to the user based on the user health graphic. In some variations, an actionable suggestion may comprise modifying computing device settings based on the user health graphic.

### EXAMPLES

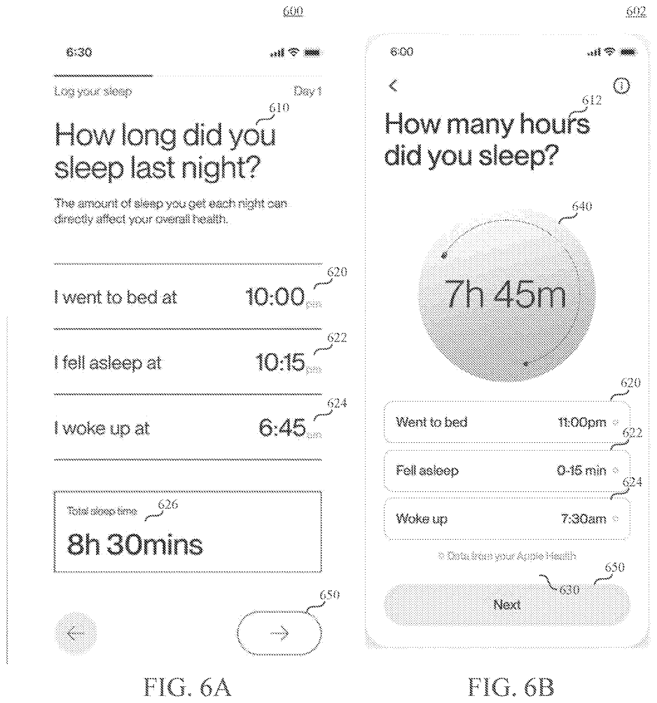

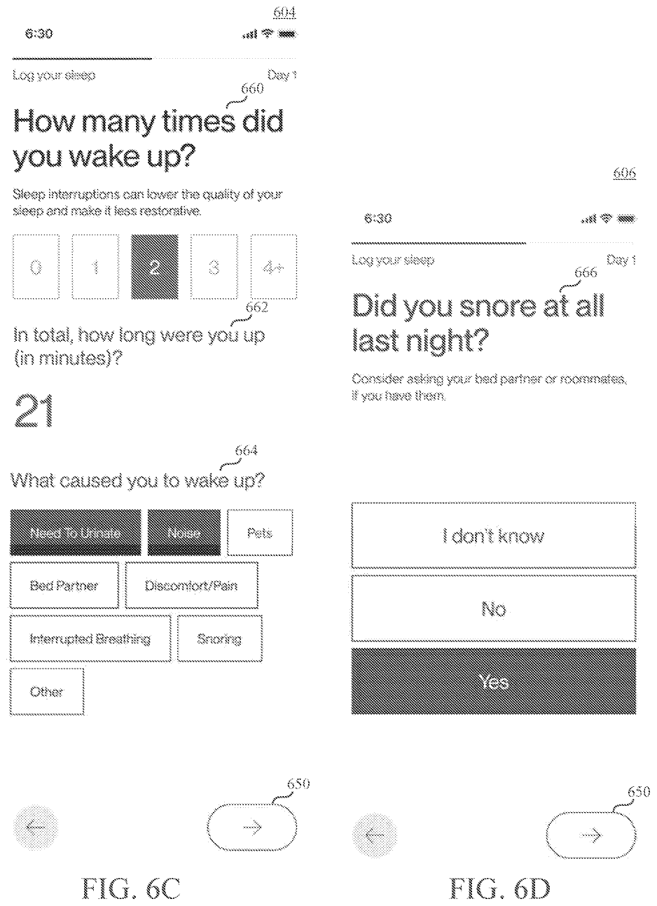

FIGS. 5A and 5B are a set of exemplary wake time graphical user interfaces (GUIs). A first GUI (500) in FIG. 5A and a second GUI (502) in FIG. 5B may include respective prompts (510, 512) configured to prepare and/or instruct a user to complete a wake assessment (e.g., morning check-in) at or soon after waking up (e.g., within 15 minutes, within 30 minutes, within 60 minutes, within 15-30 minutes, within 15-60 minutes of a wake time) on a new day (e.g., after sleep). User data collected around this time may include sleep data, physical health data, and mental health data that may be used to predict risk of one or more sleep-related disorders. If available, sleep measurement data may also be collected around this time. An advancement icon (520, 522) may be selected to advance to the wake assessment GUIs (e.g., FIGS. 6A-8). Upon completion of an assessment, a third GUI (504) in FIG. 5C and a fourth GUI (506) in FIG. 5D may include a respective prompt (514, 516) configured to indicate assessment completion and/or provide encouragement to continue completing an assessment. An advancement icon (524, 526) may be selected to advance to, for example, insight GUIs.

FIGS. 6A-6D, 7A-7H, and 8 are a set of exemplary wake assessment GUIs configured to track a wake moment based on subjective user reported outcomes and objective sleep measurement data for predicting risk of one or more sleep-related disorders. For example, a fifth GUI (600) in FIG. 6A and a sixth GUI (602) in FIG. 6B may include respective prompts (610, 612) for user sleep data. In some variations, measurement data (630) received from a measurement device may include sleep data of a user including a bedtime (620), a fall asleep time (622), a wake time (624), and total sleep time (626). These and other measured sleep parameters may be displayed and pre-populated on fifth GUI (600) and sixth GUI (602). However, any of these sleep parameters (e.g., 620, 622, 624, etc.) may be selected and modified by the user to confirm and increase the accuracy of the sleep data. In this manner, subjective user-reported sleep outcomes may be input in addition to measured sleep data from a measurement device. In some variations, the GUI may include a sleep data graphic (640) based on the sleep data. An advancement icon (650) may be selected to advance to, for example, the next GUI of the wake assessment. FIG. 6C depicts a seventh GUI (604) including a wake count prompt (660), a wake duration prompt (662), a wake reason prompt (664), and an advancement icon (650). The user may select a predetermined wake reason and/or input a wake reason. In some variations a user may select a customization icon (not shown) configured to receive user input to add one or more custom wake reasons. FIG. 6D depicts an eighth GUI (606) including a snore status prompt (666) (e.g., I don't know, no, yes) and an advancement icon (650).

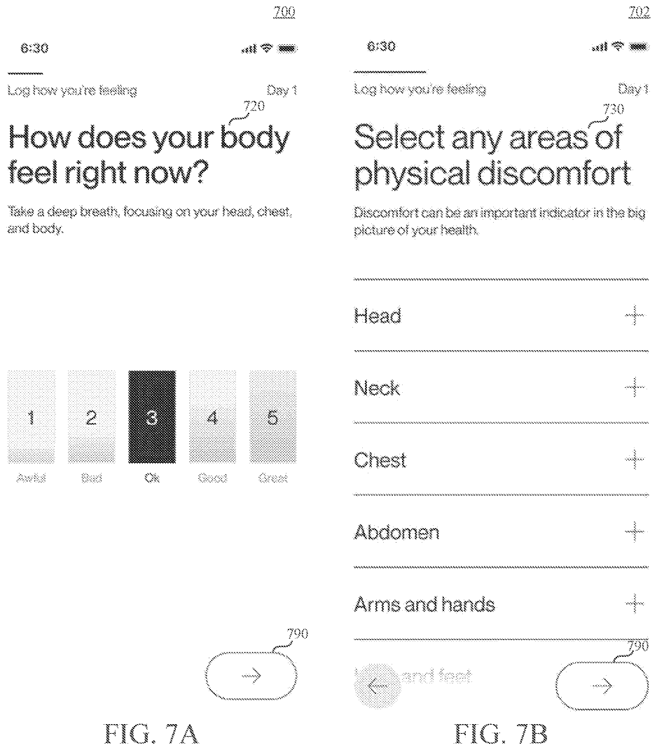

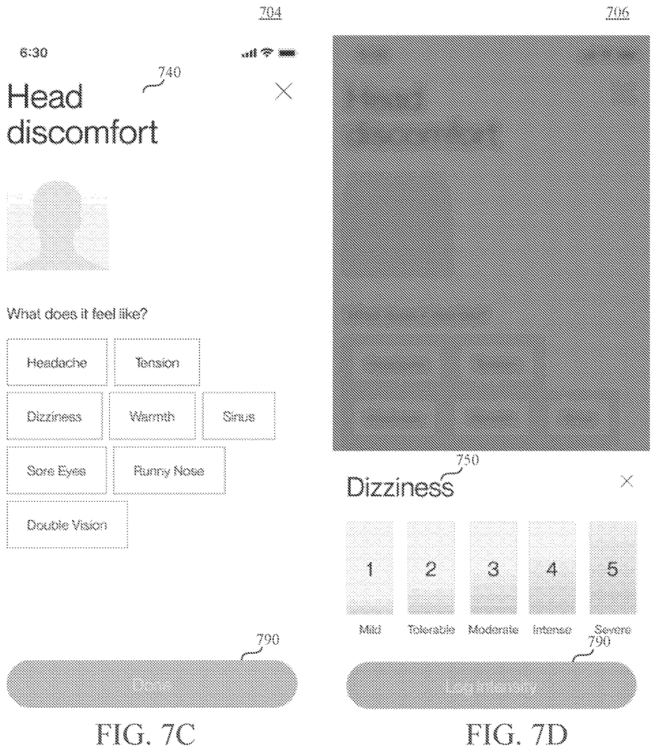

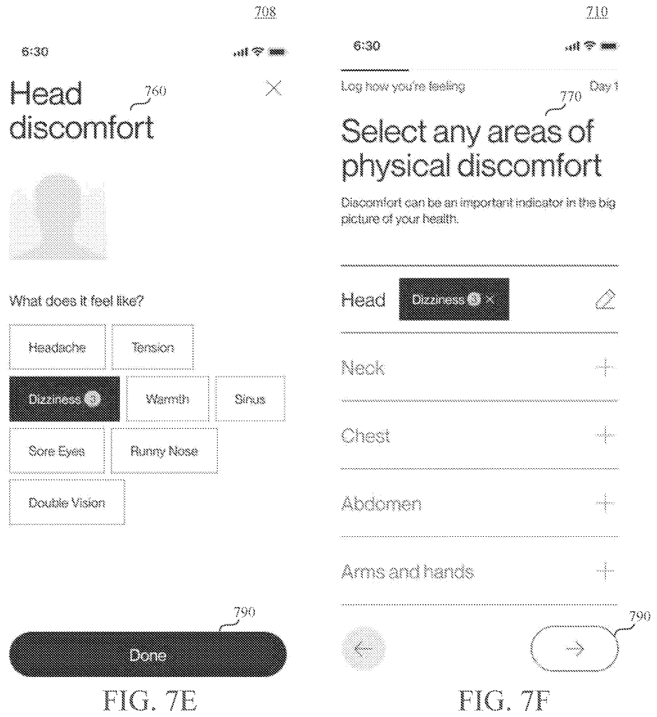

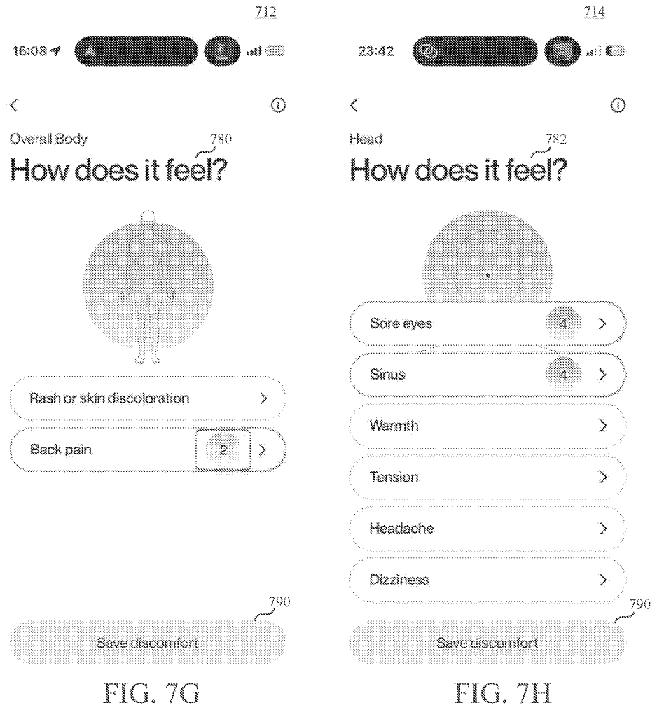

FIG. 7A depicts a ninth GUI (700) including a body status prompt (720) of a wake time assessment and an advancement icon (790). For example, a user may select a body status based on a five-point scale (e.g., awful, bad, OK, good, great). FIG. 7B depicts a tenth GUI (702) including a body discomfort area prompt (730) and an advancement icon (790). For example, the user may select one or more predetermined areas of the body that exhibit physical discomfort (e.g., head, neck, chest, abdomen, arms and hands, legs and feet). FIG. 7C depicts an eleventh GUI (704) including a body discomfort type prompt (740) and an advancement icon (790). For example, the user may select one or more predetermined types of physical discomfort (e.g., headache, tension, dizziness, warmth, sinus, sore eyes, runny nose, double vision) associated with a selected body area (e.g., head). In some variations a user may select a customization icon (not shown) configured to receive user input to add one or more custom physical symptoms and/or emotions.

FIG. 7D depicts a twelfth GUI (706) including a body discomfort intensity prompt (750) and an advancement icon (790). For example, the user may select an intensity level (e.g., on a five-point scale of mild, tolerable, moderate, intense, severe) corresponding to a selected body discomfort (e.g., dizziness) and selected body area (e.g., head). FIG. 7E depicts a thirteenth GUI (708) including a body discomfort area prompt (760) and an advancement icon (790). GUI (708) may function as a confirmation screen that the selected body discomfort area, type, and intensity is accurately reflected. FIG. 7F depicts a fourteenth GUI (710) including a body discomfort area prompt (760) including the previously input user data and an advancement icon (790). The user may include additional body discomforts as needed. FIGS. 7G and 7H depict a respective fifteenth GUI (712) and sixteenth GUI (714) including a body discomfort type prompt (780, 782) and an advancement icon (790). The GUIs (712, 714) may be configured to receive one or more of a body discomfort area (e.g., back pain), a body discomfort type (e.g., sore eyes), and a body discomfort intensity (e.g., 2, 4).

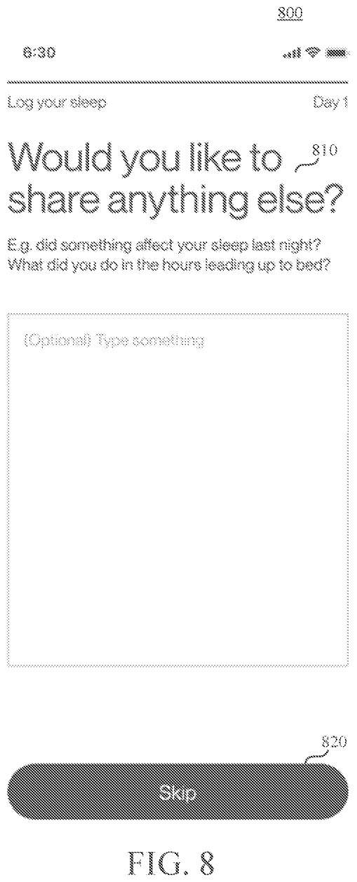

FIG. 8 depicts a seventeenth GUI (800) including a wake time note prompt (810) for optional user input and an advancement icon (820). In some variations, the wake time note may include wake time thoughts of a user that may not otherwise be captured by the sleep parameters. These thoughts may be quickly forgotten by a user if not input at a wake time. In some variations, one or more of the check-in GUIs may include a therapy and/or medication prompt for user input of one or more of a therapy the user has undertaken and/or completed and a medication a user has taken.

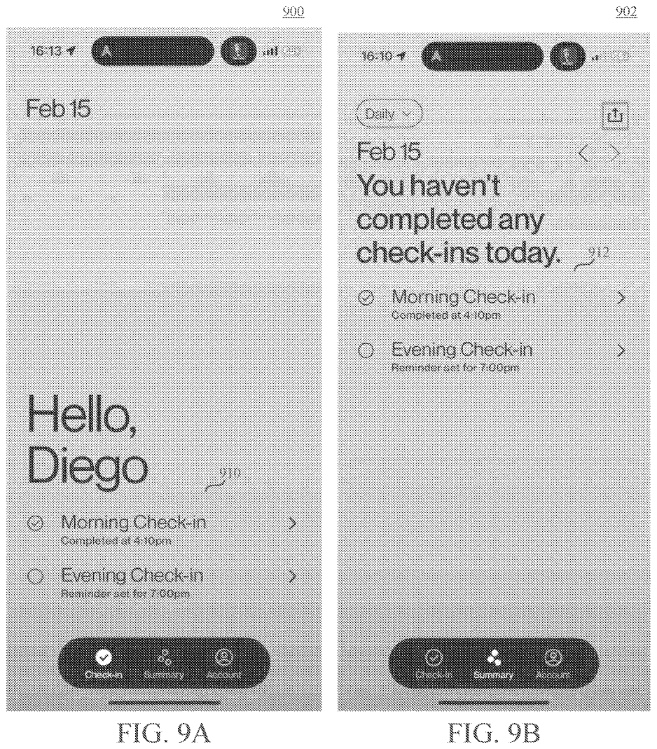

FIGS. 9A and 9B depict a respective eighteenth GUI (900) and nineteenth GUI (902) including an assessment prompt (910, 912) that may indicate an assessment status (e.g., completion, time of assessment, time of next assessment). In some variations a user may select an edit icon (not shown) configured to receive user input to edit one or more check-ins. In some variations, one or more GUIs (e.g., FIGS. 5A-5D, 9A, 9B) may include a count of, or other element (e.g., icon) representative of, consecutive check-ins performed by the user to encourage consistent input of check-ins.

FIGS. 10A-10G are a set of illustrative variations of a sleep insight GUI configured to provide trends, analysis, and insights into user sleep and health based on sleep data. The sleep insight GUIs can provide context and meaning to the sleep data that may be useful for users and health care professionals. For example, a twentieth GUI (1000) in FIG. 10A may include a prompt (1012) for a predetermined time period (1010) (e.g., daily, weekly, bi-weekly, monthly, bi-monthly, annually) corresponding to a plain language insight (e.g., trend, pattern, data analysis). The use of plain language facilitates quick, easy, and usable communication of information. A search icon (1020, 1022) (e.g., search field) may be configured to receive user input related to a specialist. For example, a user may input an inquiry via text or voice by selecting search icon (1020) in response to the prompt (1012). In some variations, a user may enter input as text or audio. A dashboard may be provided including one or more sleep trends including physical (e.g., body) discomforts (1030) configured to summarize a set of body discomfort areas (1032), body discomfort types (e.g., headache, sore eyes), and body discomfort intensities (e.g., headache intensity levels of 1, 1, 2, 3, 3, 4, and 5 through the week of January 15-21).

FIG. 10B depicts a twenty-first GUI (1002) including the dashboard of sleep trends including physical discomfort (1030), an average sleeping oxygen saturation level (1034), and an average sleeping heart rate (1036) over the predetermined time period (e.g., January 15-21). In some variations, a dashboard view may include data for a selectable predetermined time period (e.g., past week, past month, past 3 months, past 6 months, past year). FIG. 10C depicts a twenty-second GUI (1004) including a plain language prompt (1012) summarizing a user's health status based on the sleep data. A dashboard may be provided including one or more sleep trends including average sleep quality (1038). One or more sleep trends may be depicted using graphs, plots, and other visualizations. FIG. 10D depicts a twenty-third GUI (1006) including a dashboard of one or more sleep trends such as areas of physical discomfort (1040) and average time in bed (1042). FIG. 10E depicts a twenty-fourth GUI (1008) including a dashboard of one or more sleep trends such as average snoring time (1044). FIG. 10F depicts a twenty-fifth GUI (1010) including a dashboard of one or more sleep trends such as user input notes (1046) and average heart rate (1048). As described herein, the sleep trends may comprise sleep data of subjective user data and objective sleep measurement data.

FIG. 10G depicts a twenty-sixth GUI (1011) including a prompt (1012) corresponding to a summary of a trend (e.g., primary disruptor of sleep). In some variations, a plain language prompt may be followed by more detailed sleep trend data such as a dashboard including snoring data (1050) corresponding to an audio waveform recording that may be heard using playback icon (1052).

In some variations, any of the sleep insight graphical user interfaces described herein (e.g., FIGS. 10A-10G) may include a default summary view and a selectable actionable prompt providing one or more of an actionable suggestion and additional information (e.g., detail view) with respect to the prompt (e.g., prompt 1012).

FIGS. 11A and 11B are a set of illustrative variations of a prediction of a risk of a sleep disorder GUI configured to facilitate early identification of a sleep-related disorder and provide increased awareness and education of sleep related disorders, as well as recommendations for action.

FIGS. 11A and 11B depict a respective twenty-seventh GUI (1100) and twenty-eighth GUI (1102) including an average sleeping oxygen saturation level (1132) and a prediction of a risk of a sleep disorder prompt (1112), an actionable suggestion prompt (1120) for additional sleep disorder information, and an average heart rate variability (1134). In some variations, the actionable suggestion prompt (1120) may encourage a user to proactively pursue clinical action. In FIG. 11B, the actionable suggestion prompt (1120) may include a link (1140) to install a diagnostic application (e.g., an obstructive sleep apnea-diagnostic application) on the computing device.

FIG. 12 depicts a twenty-ninth GUI (1200) including an actionable suggestion prompt (1230) and a list of selectable actionable suggestions such as an earlier bedtime suggestion, snoring recording, oxygen saturation information, referral to a clinical service (e.g., specialist), and a download of an HCP report (1240). In some variations, the prompt (1230) may be generated based on the sleep data (e.g., personalized to each user) in order to facilitate improvements in sleep and health. In some variations, the actionable suggestions may encourage healthy behavior (e.g., earlier bedtime, consultation with a specialist).

FIG. 13 depicts a thirtieth GUI (1300) for facilitating a referral to a sleep service and/or other clinical action including a prompt (1340) to share a sleep and health report for a predetermined time period (1310) (e.g., weekly, monthly) with a healthcare professional. In some variations, a mobile application or web portal as described herein may generate the sleep and health report. FIGS. 14A and 14B depict a respective thirty-first GUI (1402) and thirty-second GUI (1404) including a sleep and health report including one or more sleep trends, sleep data, and user notes (e.g., wake time note, bedtime note). In some variations, one or more of a content and format of a sleep and health report may be customized by a healthcare professional. The sleep and health report may include any of the user data and sleep measurement data described herein.

In some variations, an ultrasonic audio signal may be accessible (e.g., output, graphically represented) from one or more of the sleep insight graphical user interface (e.g., FIGS. 10A-10G), sleep disorder prediction graphical user interface (e.g., FIGS. 11A-11B), referral graphical user interface (e.g., FIG. 13), and health status report (e.g., FIGS. 14A-14B, sleep and health report).

FIGS. 15A and 15B are a set of illustrative variations of a health status graphical user interface. FIGS. 15A and 15B depict a respective thirty-third GUI (1500) and thirty-fourth GUI (1502) including a first shape (1510), a second shape (1512), and a third shape (1514). A first line (1520) connects a center of the first shape (1510) to a center of a second shape (1512), and a second line (1522) connects a center of the second shape (1512) to a center of the third shape (1514). In some variations, a health status graphic may be displayed on an introduction GUI and/or in conjunction with a sleep trend and risk of sleep disorder prediction.

FIGS. 17A-17AA are a set of illustrative variations of a health status graphic configured to facilitate an intuitive and quick understanding of a user's holistic health (e.g., sleep, physical health, mental health). The health status graphic may include a first shape (1730) having a first color and a first center, a second shape (1740) having a second color and a second center, and a third shape (1750) having a third color and a third center. Optionally, a first line (1735) may connect a center of the first shape (1730) and a center of the second shape (1740), and a second line (1745) may connect a center of the second shape (1740) and a center of the third shape (1750). A first axis corresponds to a longitudinal axis of the health status graphic and a second axis corresponds to a lateral axis of the health status graphic. As discussed herein, a color may correspond to a rating on a scale (e.g., negative, neutral, positive, 1, 2, 3, 4, 5). For example, a green color may be associated with a positive rating for a parameter (e.g., sleep, physical health, mental health), yellow may be associated with a neutral rating, and red may be associated with a negative rating. In this manner, a health rating may be based on a color and/or a relative position of the shapes. For example, a health rating may be based on a color and relative position of the shapes relative to one another and/or a predetermined axis.

In some variations, as shown in FIGS. 17A-17AA, each of the first color, the second color, and the third color may comprise a color gradient. However, in other variations, one or more of the colors may not have a color gradient. In some variations, as shown in FIGS. 17A-17AA, each of the first shape (1730), the second shape (1740), and the third shape (1750) may comprise an opacity gradient. However, in other variations, one or more of the shapes may not have an opacity gradient.

In some variations, as shown in FIGS. 17A-17AA, a background of the graphical user interface may comprise a color gradient. However, the background may comprise a non-color gradient background (e.g., default background color).

FIG. 17A depicts a thirty-fifth GUI (1700) where the first shape (1730) corresponds to a positive color, the second shape (1740) corresponds to a positive color, and the third shape (1750) corresponds to a positive color. Each of the first shape (1730), the second shape (1740), and the third shape (1750) are positioned on a first axis (e.g., longitudinal axis of the GUI (1700) running through a midpoint of the GUI (1700) and not shown). Likewise, the centers of each of the first shape (1730), the second shape (1740), and the third shape (1750) are positioned on the first axis. Additionally, the first line (1735) and the second line (1745) are also positioned on the first axis. The first line (1735) and the second line (1745) each overlap two of the first shape, the second shape, and the third shape. More specifically, the first line (1735) overlaps the first shape (1730) and the second shape (1740) while the second line (1745) overlaps the second shape (1740) and the third shape (1750). In some variations, centers of two or more of the first shape, the second shape, and the third shape may be spaced apart from each other along the first axis. For example, the first shape (1730), the second shape (1740), and the third shape (1750) may partially overlap along the first axis, but the centers of the first, second, and third shapes may be positioned at different points along the first axis. In some variations, the perimeter of one shape may intersect the center of another shape, or two other shapes of the three shapes. In particular, referring to the variation depicted in FIG. 17A, a perimeter (e.g., circumference) of the second shape (1740) may intersect a center of the first shape (1730) and a center of the second shape (1750). In other variations, for example, the variation depicted in FIG. 17B, a perimeter of the second shape (1740) may intersect a center of the first shape (1730) but may not intersect the center of the third shape (1750). In yet other variations, the perimeter of the second shape (1740) may not intersect the center of either of the first shape or the third shape. In FIG. 17A, the first center, the second center, and the third center are spaced apart by an equal center-to-center distance along the first axis. Although not depicted, in some variations, all three shapes may overlap each other (whether aligned or not to an axis).

FIG. 17B depicts a thirty-sixth GUI (1701) where the first shape (1730) corresponds to a positive color, the second shape (1740) corresponds to a positive color, and the third shape (1750) corresponds to a neutral color. In FIG. 17B, the first center of the first shape (1730) and the second center of the second shape (1740) are positioned on the first axis while the third center of the third shape (1750) is offset from the first axis. Put differently, the first and second shapes (1730, 1750) are aligned, but misaligned from the third shape (1750). In this manner, the first line (1735) and the second line (1745) form a non-zero angle with respect to the first axis. FIG. 17B illustrates that the overlap of shapes is not limited. For example, the second shape (1740) and the third shape (1750) may partially overlap along an angle offset from the first axis. FIGS. 17D, 17F, 17H, 17J, 17K, and others show other examples of partial overlap of shapes.

In some variations, a center-to-center distance between the first shape and the second shape may be based on a differential between two of the sleep rating, the physical health rating, and the mental health rating. Furthermore, the color and position of the third shape (1750) relative to the other shapes indicate to the user a differential between the respective ratings of the third shape (1750) relative to the first shape (1730) and the second shape (1740). For example, the first shape (1730) and the second shape (1740) corresponds to a positive rating (e.g., positive sleep rating, positive physical health rating) while the third shape (1750) corresponds to a neutral rating (e.g., neutral mental health rating).

FIG. 17C depicts a thirty-seventh GUI (1702) where the first shape (1730) and the second shape (1740) each have a green color corresponding to a positive rating, and the third shape (1750) has a red color corresponding to a negative rating. In FIG. 17C, the first center of the first shape (1730) and the second center of the second shape (1740) are positioned on the first axis while the third center of the third shape (1750) is offset from the first axis. The first line (1735) and the second line (1745) form an angle between the first axis and a lateral axis as described herein. The color and position of the third shape (1750) indicate to the user the differential between the respective ratings of the third shape (1750) relative to the first shape (1730) and the second shape (1740). The third shape (1750) of FIG. 17C has a higher rating differential relative to the third shape (1750) of FIG. 17B.

FIG. 17D depicts a thirty-eight GUI (1703) where the first shape (1730) corresponds to a positive color, the second shape (1740) corresponds to a neutral color, and the third shape (1750) corresponds to a positive color. In FIG. 17D, the first center of the first shape (1730) and the third center of the third shape (1750) are positioned on the first axis while the second center of the second shape (1740) is offset from the first axis.

FIG. 17E depicts a thirty-ninth GUI (1704) where the first shape (1730) corresponds to a positive color, the second shape (1740) corresponds to a neutral color, and the third shape (1750) corresponds to a neutral color. In FIG. 17E, the first center of the first shape (1730) is positioned on the first axis while the second center of the second shape (1740) and the third center of the third shape (1750) are offset from the first axis. FIG. 17E depicts a variation in which the centers of two or more of the first shape, the second shape, and the third shape are offset from each other relative to the first axis.

FIG. 17F depicts a fortieth GUI (1705) where the first shape (1730) corresponds to a positive color, the second shape (1740) corresponds to a neutral color, and the third shape (1750) corresponds to a negative color. In some variations, the first center, the second center, and the third center are laterally offset from each other. The first line (1735) and the second line (1745) form an angle (e.g., about 15 degrees, about 30 degrees, about 45 degrees, about 60 degrees) between a longitudinal axis of the user health graphic and a lateral axis of the user health graphic.

FIG. 17G depicts a forty-first GUI (1706) where the first shape (1730) corresponds to a positive color, the second shape (1740) corresponds to a negative color, and the third shape (1750) corresponds to a positive color. In FIG. 17G, the first center of the first shape (1730) and the third center of the third shape (1750) are positioned on the first axis while the second center of the second shape (1740) is offset from the first axis. The color and position of the second shape (1740) indicates to the user the differential between the respective ratings of the second shape (1740) relative to the first shape (1730) and the third shape (1750). The color and position of the first shape (1730) and the third shape (1750) indicate alignment in their rating.

FIG. 17H depicts a forty-second GUI (1707) where the first shape (1730) corresponds to a positive color, the second shape (1740) corresponds to a negative color, and the third shape (1750) corresponds to a neutral color. The first center of the first shape (1730) is positioned on the first axis while the second center of the second shape (1740) and the third center of the third shape (1750) are offset from the first axis. The color and position of each of the shapes indicates to the user the differential between the ratings of each of the shapes where none of the ratings match.

FIG. 17I depicts a forty-third GUI (1708) where the first shape (1730) corresponds to a positive color, the second shape (1740) corresponds to a negative color, and the third shape (1750) corresponds to a negative color. The first center of the first shape (1730) is positioned on the first axis while the second center of the second shape (1740) and the third center of the third shape (1750) are offset from the first axis. The color and position of the first shape (1730) indicates to the user the differential between the respective ratings of the second shape (1740) and third shape (1750) relative to the first shape (1730). The color and position of the second shape (1740) and the third shape (1750) indicate alignment in their rating.

FIG. 17J depicts a forty-fourth GUI (1709) where the first shape (1730) corresponds to a neutral color, the second shape (1740) corresponds to a positive color, and the third shape (1750) corresponds to a positive color. The first center of the first shape (1730) is positioned offset from the first axis while the second center of the first shape (1740) and the third center of the third shape (1750) are positioned on the first axis. The color and position of the first shape (1730) indicates to the user the differential between the respective ratings of the first shape (1730) relative to the second shape (1740) and the third shape (1750). The color and position of the second shape (1740) and the third shape (1750) indicate alignment in their rating.

FIG. 17K depicts a forty-fifth GUI (1710) where the first shape (1730) corresponds to a neutral color, the second shape (1740) corresponds to a positive color, and the third shape (1750) corresponds to a neutral color. In FIG. 17K, the second center of the second shape (1740) is positioned on the first axis while the first center of the first shape (1730) and the third center of the third shape (1750) are offset from the first axis. The color and position of the second shape (1740) indicates to the user the differential between the respective ratings of the second shape (1740) relative to the first shape (1730) and the third shape (1750). The color and position of the first shape (1730) and the third shape (1750) indicate alignment in their rating (e.g., neutral rating).

FIG. 17L depicts a forty-sixth GUI (1711) where the first shape (1730) corresponds to a neutral color, the second shape (1740) corresponds to a positive color, and the third shape (1750) corresponds to a negative color. In FIG. 17L, the second center of the second shape (1740) is positioned on the first axis while the first center of the first shape (1730) and the third center of the third shape (1750) are offset from the first axis.

FIG. 17M depicts a forty-seventh GUI (1712) where the first shape (1730) corresponds to a neutral color, the second shape (1740) corresponds to a neutral color, and the third shape (1750) corresponds to a positive color. In FIG. 17M, the third center of the third shape (1750) is positioned on the first axis while the first center of the first shape (1730) and the second center of the second shape (1740) are offset from the first axis. The color and position of the third shape (1750) indicates to the user the differential between the respective ratings of the third shape (1750) relative to the first shape (1730) and the second shape (1740). The color and position of the first shape (1730) and the second shape (1740) indicate alignment in their rating (e.g., neutral rating).

FIG. 17N depicts a forty-eight GUI (1713) where the first shape (1730) corresponds to a neutral color, the second shape (1740) corresponds to a neutral color, and the third shape (1750) corresponds to a neutral color. When each shape has a neutral color, the shapes may be aligned at an angle (e.g., about 15 degrees, about 30 degrees, about 45 degrees, about 60 degrees, about 75 degrees) relative to vertical axis of the GUI to indicate to the neutral user health state. For example, the neutral user health state may have a positive angle (FIG. 17N) while a negative user health state may have a negative angle (FIG. 17AA). In some variations, the first axis may be an angled axis between a longitudinal axis of the user health graphic and a lateral axis of the user health graphic.

FIG. 17O depicts a forty-ninth GUI (1714) where the first shape (1730) corresponds to a neutral color, the second shape (1740) corresponds to a neutral color, and the third shape (1750) corresponds to a negative color.

FIG. 17P depicts a fiftieth GUI (1715) where the first shape (1730) corresponds to a neutral color, the second shape (1740) corresponds to a negative color, and the third shape (1750) corresponds to a positive color. In FIG. 17P, the third center of the third shape (1750) is positioned on the first axis while the first center of the first shape (1730) and the third center of the third shape (1750) are offset from the first axis.

FIG. 17Q depicts a fifty-first GUI (1716) where the first shape (1730) corresponds to a neutral color, the second shape (1740) corresponds to a negative color, and the third shape (1750) corresponds to a neutral color.

FIG. 17R depicts a fifty-second GUI (1717) where the first shape (1730) corresponds to a neutral color, the second shape (1740) corresponds to a negative color, and the third shape (1750) corresponds to a negative color. The color and position of the first shape (1730) indicates to the user the differential between the respective ratings of the first shape (1730) relative to the second shape (1740) and the third shape (1750). The color and position of the second shape (1740) and the third shape (1750) indicate alignment in their rating (e.g., negative rating).

FIG. 17S depicts a fifty-third GUI (1718) where the first shape (1730) corresponds to a negative color, the second shape (1740) corresponds to a positive color, and the third shape (1750) corresponds to a positive color. In FIG. 17S, the second center of the second shape (1740) and the third center of the third shape (1750) are positioned on the first axis while the first center of the first shape (1730) is offset from the first axis. The color and position of the first shape (1730) indicates to the user the differential between the respective ratings of the first shape (1730) relative to the second shape (1740) and the third shape (1750). The color and position of the second shape (1740) and the third shape (1750) indicate alignment in their rating (e.g., positive rating).

FIG. 17T depicts a fifty-fourth GUI (1719) where the first shape (1730) corresponds to a negative color, the second shape (1740) corresponds to a positive color, and the third shape (1750) corresponds to a neutral color. In FIG. 17T, the second center of the second shape (1740) is positioned on the first axis while the first center of the first shape (1730) and the third center of the third shape (1750) are offset from the first axis on opposite sides of the axis.

FIG. 17U depicts a fifty-fifth GUI (1720) where the first shape (1730) corresponds to a negative color, the second shape (1740) corresponds to a positive color, and the third shape (1750) corresponds to a negative color. In FIG. 17U, the second center of the second shape (1740) is positioned on the first axis while the first center of the first shape (1730) and the third center of the third shape (1750) are offset from the first axis on the same side of the axis.

FIG. 17V depicts a fifty-sixth GUI (1721) where the first shape (1730) corresponds to a negative color, the second shape (1740) corresponds to a neutral color, and the third shape (1750) corresponds to a positive color. In some variations, the first axis may be an angled axis (e.g., having a negative slope) between a longitudinal axis of the user health graphic and a lateral axis of the user health graphic.

FIG. 17W depicts a fifty-seventh GUI (1722) where the first shape (1730) corresponds to a negative color, the second shape (1740) corresponds to a neutral color, and the third shape (1750) corresponds to a neutral color. The color and position of the first shape (1730) indicates to the user the differential between the respective ratings of the first shape (1730) relative to the second shape (1740) and the third shape (1750). The color and position of the second shape (1740) and the third shape (1750) indicate alignment in their rating (e.g., neutral rating).

FIG. 17X depicts a fifty-eighth GUI (1723) where the first shape (1730) corresponds to a negative color, the second shape (1740) corresponds to a neutral color, and the third shape (1750) corresponds to a negative color. The color and position of the second shape (1740) indicates to the user the differential between the respective ratings of the second shape (1740) relative to the first shape (1730) and the third shape (1750). The color and position of the first shape (1730) and the third shape (1750) indicate alignment in their rating (e.g., negative rating).

FIG. 17Y depicts a fifty-ninth GUI (1724) where the first shape (1730) corresponds to a negative color, the second shape (1740) corresponds to a negative color, and the third shape (1750) corresponds to a positive color. In FIG. 17Y, the third center of the third shape (1750) is positioned on the first axis while the first center of the first shape (1730) and the second center of the second shape (1740) are offset from the first axis.

FIG. 17Z depicts a sixtieth GUI (1725) where the first shape (1730) corresponds to a negative color, the second shape (1740) corresponds to a negative color, and the third shape (1750) corresponds to a neutral color.

FIG. 17AA depicts a sixty-first GUI (1726) where the first shape (1730) corresponds to a negative color, the second shape (1740) corresponds to a negative color, and the third shape (1750) corresponds to a negative color. In some variations, the first axis may be an angled axis (e.g., having a negative slope) between a longitudinal axis of the user health graphic and a lateral axis of the user health graphic.

The foregoing descriptions, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. A method of graphically representing user health, comprising:
receiving user data comprising a sleep rating, a plurality of physical health parameters, and a mental health rating from a computing device and sleep measurement data from a measurement device;
generating a sleep quality rating based on the sleep rating and the sleep measurement data, a physical health quality rating based on the plurality of physical health parameters, and a mental health quality rating based on the mental health rating;
generating a user health graphic on a graphical user interface of the computing device, wherein the generating comprises:
generating a first shape based on the sleep quality rating, a second shape based on the physical health quality rating, and a third shape based on the mental health quality rating; and
arranging a location of the first shape, the second shape, and the third shape relative to a first axis of the user health graphic based on the sleep quality rating, the physical health quality rating, and the mental health quality rating.

2. The method of claim 1, further comprising periodically generating the sleep quality rating, the physical health quality rating, and the mental health quality rating.

3. The method of claim 1, wherein the sleep measurement data comprises one or more of sleep duration, sleep efficiency, sleep latency, a number of awakenings, time spent awake, and sleep quality.

4-5. (canceled)

6. The method of claim 1, wherein the plurality of physical health parameters comprises two or more of a physical health rating, a number of physical discomforts, a number of maximum value physical discomforts, and an average severity of physical discomfort.

7-10. (canceled)

11. The method of claim 1, wherein generating the user health graphic comprises an alphanumeric representation of the sleep quality rating, the physical health quality rating, and the mental health quality rating.

12-13. (canceled)

14. The method of claim 1, wherein the first axis is a longitudinal axis of the user health graphic.

15. The method of claim 1, wherein the first shape comprises a first center, the second shape comprises a second center, and the third shape comprises a third center, wherein the first center, the second center, and the third center are positioned on the first axis.

16. The method of claim 1, wherein the first shape comprises a first center, the second shape comprises a second center, and the third shape comprises a third center, wherein two of the first center, the second center, and the third center are positioned on the first axis, and a third of the first center, the second center, and the third center is offset from the first axis.

17. The method of claim 1, wherein the first shape comprises a first center, the second shape comprises a second center, and the third shape comprises a third center, wherein one of the first center, the second center, and the third center are positioned on the first axis, and a remaining two of the first center, the second center, and the third center are offset from and on opposing sides of the first axis.

18. The method of claim 1, wherein the first axis is an angled axis between a longitudinal axis of the user health graphic and a lateral axis of the user health graphic.

19. The method of claim 1, wherein a center-to-center distance between the first shape and the second shape is based on a differential between two of the sleep rating, the physical health quality rating, and the mental health rating.

20. The method of claim 1, wherein centers of two or more of the first shape, the second shape, and the third shape are spaced apart from each other along the first axis.

21. The method of claim 1, wherein centers of two or more of the first shape, the second shape, and the third shape are offset from each other relative to the first axis.

22. The method of claim 1, wherein the first shape comprises a first center, the second shape comprises a second center, and the third shape comprises a third center, wherein the first center, the second center, and the third center are laterally offset from each other.

23. The method of claim 22, wherein the first shape, the second shape, and the third shape partially overlap along the first axis.

24. The method of claim 1, wherein the first shape comprises a first center, the second shape comprises a second center, and the third shape comprises a third center, wherein the first center, the second center, and the third center are spaced apart by an equal center-to-center distance along the first axis.

25. The method of claim 1, wherein generating the user health graphic comprises generating a plurality of lines, wherein each line of the plurality of lines overlaps two of the first shape, the second shape, and the third shape.

26. The method of claim 25, wherein the plurality of lines comprises a first line between a center of the first shape and a center of the second shape, and a second line between the center of the second shape and a center of the third shape.

27. (canceled)

28. The method of claim 1, wherein each of the first shape, the second shape, and the third shape comprises at least one of: (i) a color gradient or (ii) an opacity gradient.

29. (canceled)

30. The method of claim 1, wherein a background of the graphical user interface comprises a color gradient.

31. (canceled)

32. The method of claim 1, further comprising generating an animation comprising an updated user health graphic and one or more previous user health graphics.

33. (canceled)

34. The method of claim 1, further comprising modifying computing device settings based on the user health graphic.

35. (canceled)

36. The method of claim 1, comprising:
predicting a risk of one or more sleep disorders based on the user data and the sleep measurement data; and
referring a sleep service to a user based on the predicted risk of the one or more sleep disorders.

37. (canceled)

38. The method of claim 1, wherein the sleep measurement data comprises one or more sleep parameters, and wherein the one or more sleep parameters comprise one or more of a sleep start time, a sleep end time, a sleep duration, a sleep stage, a time to fall asleep, a number of times woken, a wake period duration, a snore status, a snore duration, an exercise duration, an exercise end time, a number of alcoholic drinks consumed, a time of last alcoholic drink consumed, a number of caffeine drinks consumed, a time of last caffeine drink consumed, a daytime sleepiness status, a daytime sleepiness severity, a daytime sleepiness status, a daytime sleepiness severity, an average sleeping oxygen saturation, an average sleeping heart rate variability, a minimum sleeping heart rate, a maximum sleeping heart rate, an average sleeping heart rate, a set of medications consumed, user demographic data, a user wake time note, or a user bedtime note.

39-42. (canceled)

43. The method of claim 6, wherein the sleep rating comprises a sleep quality status, the physical health rating comprises a body status, and the mental health rating comprises an emotional status.

44. The method of claim 43, wherein the sleep quality status comprises a wake reason.

45. The method of claim 43, wherein the body status comprises a body discomfort type a body discomfort area, and a body discomfort intensity.

46. The method of claim 43, wherein the emotional status comprises a wake time emotion and a bedtime emotion.

47-51. (canceled)

52. The method of claim 36, further comprising modifying computing device settings based on one or more of a sleep trend and the risk of the one or more sleep disorders.

53-56. (canceled)

57. The method of claim 36, wherein the one or more sleep disorders comprise one or more of obstructive sleep apnea, central sleep apnea, hypoapnea, orthopnea, nocturnal atrial fibrillation, nocturnal hypertension, nocturnal discomfort, evening exacerbated chronic obstructive pulmonary disease, heart failure, asthma, sleep quality, and waking discomfort.

58-107. (canceled)
